(19) United States
(12) Patent Application Publication
(10) Pub. No.: US 2026/0224447 A1
(43) Pub. Date: Aug. 6, 2026

(54) FORMULATIONS OF LIPOPHILIC SUBTANCES FOR COSMETIC USES

(71) Applicant: KARNAK TECHNOLOGIES, LLC, New York, NY (US)

(72) Inventor: Rafael Ezra, Tel Aviv (IL)

(21) Appl. No.: 19/553,108

(22) Filed: Feb. 27, 2026

Related U.S. Application Data
(63) Continuation of application No. 17/800,333, filed on Jul. 29, 2021, now abandoned.
(60) Provisional application No. 63/058,255, filed on Jul. 29, 2020.

(51) Int. Cl. A61K 8/02; A61K 8/11; A61K 8/60; A61K 8/67; A61K 8/73; A61K 8/92; A61Q 17/04; A61Q 19/08

(52) U.S. Cl. CPC A61K 8/022; A61K 8/11; A61K 8/60; A61K 8/67; A61K 8/731; A61K 8/922; A61Q 17/04; A61Q 19/08; A61K 2800/412; A61K 2800/413

(57) ABSTRACT

The invention generally provides a range of compositions and methods that are highly applicable to the field of cosmetics. The invention applies a nanotechnological approach to produce a new generation of compositions providing an improved topical and dermal delivery of lipophilic actives, and specifically non-therapeutic actives that serve as a basis for a range of skin care, personal care, and fragrance products.

### CROSS-REFERENCE TO RELATED APPLICATIONS

This application is a continuation of and claims the benefit of U.S. patent application Ser. No. 17/800,333, filed Aug. 17, 2022, pending, which is a National Phase Entry of PCT International Application No. PCT/IL2021/050917 which was filed on Jul. 29, 2021, which claims priority to U.S. Provisional Patent Application Ser. No. 63/058,255, filed Jul. 29, 2020, all of which are hereby incorporated by reference in their entireties.

### TECHNOLOGICAL FIELD

The invention generally relates to topical and dermatological compositions of lipophilic actives, and specifically non-therapeutic actives with cosmetic benefits that can serve as a basis for a range of skin, hair, and personal care products.

### BACKGROUND

The cosmetic industry is constantly seeking new and pioneering products that will combine both, proven biological activity and an efficient delivery of actives. Although conventional formulations are still the primary cosmetic products seen on the market, numerous advances have been made in the development of newer and innovative dermal delivery systems for cosmetics.

Recently, micro- and nanoemulsions have emerged as prospective delivery systems to overcome the limitations of the existing conventional macroemulsions that, for the most part, are thermodynamically unstable and have a relatively limited shelf life. Nanoemulsions are particularly attractive for pharmaceuticals, foods and cosmetics due to a range of advantageous properties, revealed in a lower content of surfactants, relative stability, lack of toxicity or irritant characteristics, low viscosity, translucent appearance, and adaptability to various formulations such as foams, creams, liquids and sprays.

In terms of cosmetic applications, the nanoemulsions provide additional advantages. Because of a small droplet size, the nanoemulsions provide a closer contact with the stratum corneum (SC) thereby increasing the amount of actives reaching the target site, improving the skin penetration, and enhancing the active efficacy, overall.

Despite the apparent advantages of nanoemulsions, some studies point to certain instability mechanisms resulting from Ostwald ripening or coalescence. Nanoemulsions are classified based on their morphology as ‘water-based’ or oil-in-water (O/W) emulsions having oil as the dispersed phase in water, or as ‘oil-based’ or water-in-oil (W/O) emulsions having the inversed morphology. The O/W nanoemulsions, in particular, exhibit growth in particle size and heterogeneity in particle size over time due to Ostwald ripening effect.

Other factors are the specific composition of constituents of the nanoemulsions, and the method of preparation. For example, the preparation of nanoemulsion usually considers hydrophile-lipophile balance (HLB), a semi-empirical value reflecting the balance of the size and strength of the hydrophilic and lipophilic moieties of surfactants, to ensure emulsification and to prevent flocculation or coalescence. Preparation of O/W nanoemulsions will usually require polymeric insoluble surfactant to stabilize against Ostwald ripening.

Further, the conditions that highly favor the formation of nanoemulsions is when surfactants are first mixed with the oily phase. In contrast, mixing surfactants with water in the initial stages of preparation, usually favors the development of ‘macroscopic’ emulsions. Another important factor is the emulsification technique. The non-equilibrium nature of nanoemulsions means that they cannot be prepared spontaneously but require sufficient mechanical energy. High energy methods such as high shear stirring with a rotor/stator system, ultrasonication, high-pressure homogenization, micro-fluidization, are instrumental for obtaining a smaller and uniform particle size.

In other words, there are specific problems with producing nanoemulsions and the O/W nanoemulsions, in particular. This latter makes the nano-emulsification of lipophilic substances particularly challenging.

Lipophilic substances and bioactives are predominant constituents of many cosmetic products. Natural oils, lipophilic antioxidants, vitamins, polyphenols and flavonoids are only some examples. Another example is Vitamin A (retinol)-retinol containing creams are the most popular on the market, and so are its derivates (retinoid, retinaldehyde and tretinoin). More recent anti-aging and exfoliation products use enzymes, many of which are lipophilic. Therefore, nano-emulsification of lipophilic actives poses a specific challenge to the field of cosmetics.

There are several precedents in this field: Mayer et al reported on successful encapsulation of vitamin E in a nanoemulsion (Mayer et al 2013, J Colloid Interface Sci 402:122-130); US 2013/0095157 suggested a method for stabilizing retinol by nano-emulsification; JP 2005/206567 suggested a method for formulating dry collagen (a hydrophobic molecule) as a nanoparticle powder; EP 1327434 described a nanoemulsion with the main metabolites of ginseng saponin. None of them however suggested a holistic formulation approach that can be applicable to various cosmetic lipophilic actives.

### General Description

The market for cosmetics and cosmeceutical, a hybrid of cosmetics and pharmaceutical products, has grown substantially in recent decades following increasing consumer awareness for dermatologically nutritional products that promote skin health and prevent skin diseases. The applications of cosmetic products have now extended to skin protection, whitening, tanning, anti-aging and anti-wrinkling, alongside other uses such as for nail and hair care, personal care, and deodorants.

Nanotechnology is an area of rising attention that unwraps new possibilities for the cosmetic industry. Nanotechnology is superior to the conventional formulation technologies as regards capabilities to produce products with enhanced characteristics, a better quality and safety, and increased shelf life. Today, nanomaterials serve as a basis for qualitative and quantitative production of foods and drugs with enhanced qualities and new types of functionalities.

With respect to poorly water-soluble or lipophilic actives, nano-delivery systems such as nanoemulsions, dendrimers, nano-micelles, solid lipid nanoparticles using specific solubility enhancers, provide promising strategies for improving solubility, permeation, release, and delivery of lipophilic actives, overall. Some of these systems further provide prolonged or controlled delivery of actives.

The increased demand for cosmetic nanoemulsions is a direct outcome of the above. Active ingredients in the form of nanoemulsions or nanoparticles have a higher surface-to-volume ratio, which enables to optimize their dispersibility and applicability for cosmetics. Due to the submicronic particles size, destabilization phenomena such as creaming, sedimentation, flocculation and coalescence that are characteristic of macroemulsions, are also avoided. These, together with the potential for controlled or prolonged release of actives, makes the nanoemulsions better adapted for cosmetic applications.

The present invention makes part of such emerging new technologies.

The primary focus of the invention has been to explore novel strategies for resolving problems associated with nanoemulsification of lipophilic substances and improving their performance in cosmetic products, such as cosmetically beneficial oils, lipophilic vitamins, enzymes and antioxidants, organic UV filters, plant, and animal extracts, and organic alcohol-free perfumes.

To that end, the invention provides an exclusive formulation approach to improving solubility and permeations of lipophilic actives, and thereby improving their topical and dermal bioavailability vivo. Importantly, as has been presently demonstrated, the formulation approaches of the invention are compatible with many kinds of cosmetic lipophilic active, and therefore have the potential of wide-ranging cosmetic applications.

The compositions of the invention constitute a solid microparticulate matter which is fully dispersible in water. This quality, per se, constitutes a significant advantage in terms stability, storage, operability, and applicability to cosmetic.

Other properties of the present compositions reside in the specific composition and arrangement of its core components, i.e., the sugars, the polysaccharides, the surfactants and the lipophilic nanospheres containing cosmetic active ingredients in acceptable oil carriers. The present studies show that the oils and actives can be distributed inside and outside the lipophilic nanospheres, which is responsible for the feature of differential bioavailability characteristic of the present compositions. The sugars, polysaccharides, and surfactants provide a formation or a porous mesh entrapping the lipophilic nanospheres. The formation or the porosity of the mesh can be modulated by the relative content of sugars, polysaccharides, surfactants, and oils, and the size of lipophilic nanospheres, which in turn impacts on the microparticulate structure and texture of the matter as a whole. Advantages of this particular structure have been revealed in surprising features of preservation of particles size upon dispersion in water, long-term stability, high loading capacity characteristic of the compositions of the invention.

Specific examples of the core components of the present compositions are trehalose, sucrose, mannitol, lactitol and lactose as sugars; maltodextrin and carboxymethyl cellulose (CMC) as polysaccharides; and ammonium glycyrrhizinate, pluronic F-127 and pluronic F-68 as surfactants. In terms of oil carriers, the compositions of the invention can use natural and synthetic oils, and specifically cosmetic oils. In terms of cosmetic lipophilic actives, many substances regulated under FD&C Act (Federal Food, Drug, and Cosmetic Act) and DSHEA (the amended Dietary Supplement Health and Education Act) and generally classified as GRAS (Generally Recognized as Safe) are plausible candidates for the compositions of the invention.

Thus, the present compositions are essentially hybrid formulations combining the advantages of lipid-based formulations and nanoparticles in terms of high loading, long-term stability, reproducibility, dermal and topical bioavailability, and other properties. All these structural and functional properties of the present compositions have been presently explored and exemplified.

More specifically, the key feature of preservation of the nanometric particle size upon reconstitution of the powder compositions in water was found to be consistent throughout various processes of production, storage conditions and various composition of sugars, oils, and lipophilic actives, combination of actives, and complex mixes of actives as in natural extracts (EXAMPLES 1-2).

First, the feature of reproducible nanometric size of the lipophilic nanospheres is highly surprising, especially in view of the known tendency of the nanoemulsion to increase particle size or fuse under various conditions. Second, it is compatible with the cosmetic applications which usually involve dispersion and dilution of actives. Third and the most important, it suggests that the benefits of nanonization can be preserved in the on the skin and the dermal tissue(s), with the expected consequences of high topical and dermal bioavailability and permeation of actives in situ.

Overall, it can be stated that the compositions of the invention provide consistent loading, entrapment, preservation, and reconstitution capacities of lipophilic actives that are preserved through various exposures, manipulations, and conditions.

The feature of chemical preservation of actives was addressed in a study showing that the compositions of the invention prevented degradation and oxidation of actives, even with actives sensitive to increased temperature, pro-oxidative species, and acidic pH such as lycopene and fish oil (EXAMPLE 2).

The feature of high loading capability was further addressed in a study showing that the compositions of the invention can be loaded with lipophilic actives and oil carriers up to 90%-95% of the initial weight of the oil component (w/w), this is without disrupting the core characteristics of preservation of nanometric particle size of the reconstituted powder (EXAMPLE 3).

Another important feature is improved topical and dermal delivery and bioavailability of actives. This feature has been demonstrated in two independent studies in an animal model using two different types of lipophilic actives, the cannabinoids and Vitamin D3, whereby the compositions of the invention exhibited advantageous patterns of immediate and/or prolonged release of actives into the circulation and tissues (EXAMPLE 4).

The feature of improved bioavailability and its specific application to cosmetics was further corroborated in studies demonstrating improved topical and dermal delivery and permeation of lipophilic actives in the compositions of the invention into various layers of human skin and the epidermis as a whole (EXAMPLE 6).

Moreover, while being exposed to high osmolarity mimicking the conditions of the human skin, the compositions of the invention preserved their core characteristics of preservation of particle size (EXAMPLE 1.8). The same was true for the polymeric PVA films, wherein the compositions of the invention have proved to be consistent in terms of uniformity and preservation of particle size upon dissolution and release from the film (EXAMPLE 5). This latter is particularly advantageous since polymeric films with embedded actives can provide attractive dosage forms for topical and dermal applications.

Still another important feature of the compositions of the invention resides in different distributions lipophilic actives and oil carriers inside and outside the lipophilic nanospheres, and the ability to increase the encapsulation capacity (EXAMPLES 1.6-1.7). This feature is highly useful in providing compositions with differential bioavailability of the entrapped and the non-entrapped actives. It is further responsible for the bi-phasic release profile combining immediate and/or prolonged release profiles of actives characteristic of the compositions of the invention (EXAMPLE 4).

It can be stated that the immediate and/or prolonged release of actives are essential attributes of the present compositions, per se, as they arise from the specific composition and structure of their core components. Overall, these features are reflected in improved topical and dermal bioavailability of the present compositions over lipid forms with the same actives.

Importantly, the compositions of the invention permit modulation of release profiles by controlling the distribution of lipophilic actives and oil carriers inside and outside the lipophilic nanospheres and thereby controlling the encapsulation capacity. Encapsulation of lipophilic actives is dependent on the amounts and types of oil carriers and/or the amount and types of sugars, polysaccharides, and surfactants. It can be further enhanced by removal of the non-encapsulated oil with hexane, for example.

In other words, the amount and/or the proportion of oil carriers and other components govern the structure and the entrapment capacity of the lipophilic actives, which in turn governs their differential availability. Thus, the loading, encapsulation capacity and bioavailability of lipophilic actives can be modulated by varying the amounts and proportions of the core components of the compositions.

In practical terms, the compositions of the invention can include various distributions and ratios of lipophilic actives and oil carriers inside or outside the lipophilic nanospheres up to the extent of ratios between about 1:0 to 9:1, respectively, and specifically as ratios between about 4:1, 7:3, 3:2, 1:1, 3:7 or 1:4, respectively.

This combination of features, i.e., solid, or semisolid consistency, the dispersibility in water, the preservation of nanometric particles size under various conditions, the ability to protect actives against harmful exposures during production and storage, the immediate and/or prolonged release of lipophilic actives and the improved topical and dermal bioavailability, make the present compositions particularly attractive as a base for formulating lipophilic substances for various cosmetic applications.

In addition, the method of preparation of the present compositions is compatible with various technologies in terms of homogenization and drying, in other words, it is not strictly bound to a single method.

It should be appreciated that this combination of properties is an essential attribute of the present compositions, per se, as it arises from the structure and composition of their core components. Interestingly with the present compositions, it is feasible to improvise and modulate these properties by varying the amounts and the proportions of their core components, like the amounts cosmetic oil(s), lipophilic actives, lipophilic nanospheres, and/or the amounts and types of sugars, polysaccharides, and surfactants.

It should be noted that cosmetic and cosmeceutical products can include additional components such as carriers, excipients, or additives for the enhancement of specific properties such as specific targeting and other properties of viscosity, texture, translucency, a tincture of color and smell, etc.

Overall, the presently proposed formulation approach offers a substantial degree of flexibility and applicability to numerous types of cosmetic products. The invention applies a nanonization technology to make and manipulate matter on a new size scale for producing novel structures with highly unique properties and wide-ranging applications.

Ultimately, the powder compositions of the invention have been related to properties of higher loading, higher encapsulation capacity, higher stability, modulated release and improved topical and dermal bioavailability, which significantly exceeded those related to analogous lipid-based compositions; this, with a minimum concentration of surfactants. In addition, in contrast to lipid-based compositions where there is a limited play with excipients, the compositions of the invention permit application of a full range of excipients. All these make the compositions of the inventions a promising approach for designing and developing unique products with high commercial potential and edge in a very competitive and lucrative global cosmetic market.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings.

FIG. 1 illustrates the feature of preservation of particle size characteristic of the powder compositions of the invention. Figure shows powder compositions comprising cannabinoids (THC or CBD) stored at 45° C. (oven) for 1, 35, 54, 72 and 82 days (3 months correlates to 24 months at RT).

FIG. 2 illustrates the feature of protection of lipophilic actives imparted by the powder compositions of the invention. Figure shows TOTOX (overall oxidation state) values for fish oil (solid line) and the powder formulation comprising the same (dashed). Fish oil is sensitive to oxidation. Figure shows significantly lower levels of the primary and secondary oxidation products in the fish oil formulated into the powder composition starting from day 0 and up to day 14.

FIGS. 3A-3B illustrate the feature of improved tropical and dermal bioavailability characteristic of the present powder compositions (LL-P) compared to the lipid forms (LL-OIL) with the same actives, CBD (3A) and THC (3B), upon administration of a single oral dose in a rat model. Figures show that the LLPs actives release profiles in plasma are significantly boosted compared to LL-OILs, and further, that LLPs exhibit an advantageous bi-phasic profiles conferring immediate and prolonged actives release.

FIGS. 4A-4D show that the advantages of improved tropical and dermal bioavailability characteristic of the powder compositions are not only specific to plasma but can be reproduced in other tissues. Figures show that in the liver and brain of the animals as above, LL-Ps exhibited similar bi-phasic release profiles of actives (THC and CBD), with an overall significantly increased release of actives compared to LL-OILs.

FIG. 5 shows that the advantages of improved tropical and dermal bioavailability can be reproduced with other lipophilic actives. Figure shows plasma levels of vitamin D3 when formulated in the powder composition of the invention (solid line) compared to the lipid composition with the same active (dashed), upon single oral dose administration in a rat model, with a 2-fold increased release of vitamin D3 in the powder composition over the analogous lipid composition.

FIG. 6 illustrates the advantage of improved permeation through the skin characteristic of the compositions of the invention as revealed in ex vivo model of human skin. Figure shows permeation of vitamin A in the powder composition vs. the analogous lipid form, with an increased permeation into the outer layer of the skin (stratum corneum), and a 6-fold increased permeation of vitamin A into the epidermis of the powder composition over the lipid form.

FIGS. 7A-7C show that the advantages of improved permeation can be reproduced with other lipophilic actives. Figures show studies in the same experimental system comparing CBD permeation in the powder and lipid compositions, with unequivocal evidence of increased CBD permeation into the 1st and 2nd layers of the stratum corneum (7A) and into the epidermis overall (4B) and a significantly higher cumulative transport of CBD through the skin (4C) in the powder composition over the analogous lipid form.

### DETAILED DESCRIPTION OF EMBODIMENTS

It should be appreciated that the invention is not limited to specific methods, and experimental conditions described herein, and that the terminology used herein is for the purpose of describing specific embodiments is not intended to be limiting.

The increased consumer awareness towards hazards related with sun exposure and aging has given a boost to cosmetics industry with an expanding variety of sophisticated compositions of bioactive compounds. Plant extracts, for example, are sources of active ingredients that function as UV protection, whitening, tanning, exfoliating, anti-wrinkling, and antiaging agents, among others. Nowadays, there is a growing demand in agro-industrial byproducts for applications in sun care and antiaging products.

Many of these agents are lipophilic and thus, the cosmetic industry is currently developing various delivery systems to increase the availability or skin permeation of lipophilic bioactive agents, such as phytochemicals, vitamins, antioxidants, essential oils, enzymes and various extracts of animals and plants. Due to their poor solubility, there are significant challenges associated with incorporating these different bioactives into cosmetic products and readily consumable forms. Different nanoemulsion fabrication methods have been employed for improving the stability and permeability of various kinds of hydrophobic vitamins and enzymes.

One of the main disadvantages of nanoemulsions is their relative instability in terms of particles size over time. Particularly the nanoemulsions in solid powder forms, suffer from this lack of uniformity in particle size, and particularly after reconstitution in water. In addition, with nanoemulsions there is a tendency to increase particle size due to fusion or reconstruction of nanoparticles.

Increased particle size and lack of uniformity leads to significant variability in the absorption of substances entrapped in the nanoparticles, and poor bioavailability. The larger particles have smaller surface area, and as a result, an inferior absorption in tissues. In the context of skin, changing conditions of osmolarity, salinity and dryness can may have significant impact on the particle size of the nanoemulsions. Therefore, despite promising prospects offered by the nanoemulsion technology, there are still significant drawbacks with its incorporation into the cosmetic industry.

The present invention has proved to surpass these difficulties in providing nanonized powder formulations of lipophilic substances, which while being readily dispersible in water preserve properties of loading, entrapment and storage potential and improved bioavailability.

In the broadest sense, the compositions of the invention can be articulated as solid water-dispersible compositions of cosmetic lipophilic active ingredients per se or in combination with cosmetic oils. Importantly, due to the solid or semisolid constitution and the ability to produce homogenous dispersions in water, the present compositions are especially advantageous for long-term storage and applicability to various high-throughput manufacturing technologies.

In numerous embodiments the compositions of the invention are provided a form of water-dispersible powders.

With respect to the actives, the term “cosmetic lipophilic active ingredient” herein is an umbrella term for a wide range of substances used in the context of cosmetics. According to the FDA, cosmetics are defined as ‘intended to be applied to the human body for cleansing, beautifying, promoting attractiveness, or altering the appearance without affecting the body's structure or functions’.

In this connection, active ingredients that are applicable to the invention do not include conventional therapeutic actives, strictly pharmaceutical products or actives, or human drugs regulated under FDA or EMA and/or provided under prescription by health care professionals.

In numerous embodiments the cosmetic lipophilic active ingredients that are applicable to the invention can be classified on the basis of their chemical structure, compositions and/or chemic activity, such as peptides, polysaccharides, phenolic substances, aromatic substances, enzymes, nucleic acids, phytochemicals, vitamins, antioxidants, essential oils, natural extracts of animal from plant sources, or combinations thereof.

In other embodiments the applicable cosmetic lipophilic active ingredients can be classified on the basis of their functionality in the context of cosmetics, such as UV protection agents, moisturizing agents, whitening agents, tanning agents, anti-wrinkling agents, elasticity promoting agents, antiaging agents, skin exfoliation agents, or combinations thereof.

It should be appreciated that a given lipophilic active ingredient can belong to more than one of these groups.

More broadly, the term “cosmetic lipophilic active ingredient” implies any active having cosmetic benefits to the skin, hair and/or nails.

By another definition, the relevant candidates can include any substance regulated under FD&C Act (Federal Food, Drug, and Cosmetic Act) and/or

DSHEA (the amended Dietary Supplement Health and Education Act) that are classified as GRAS (Generally Recognized as Safe) and are generally characterized as lipophilic.

The term “lipophilic” requires additional attention. Lipophilicity refers to the ability of a chemical compound to dissolve in fats, oils, lipids, and non-polar solvents. Lipophilicity, hydrophobicity, and non-polarity describe the same tendency, although they are not synonymous. Lipophilicity of uncharged molecules can be estimated experimentally by methods measuring the partition coefficient (log P) in a water/oil biphasic system. For molecules that are weak acids or bases, the measurements must further consider the pH wherein the majority of species remain uncharged. A positive value for log P denotes a higher concentration in the lipid phase.

Thus, in numerous embodiments, the invention applies to uncharged or weekly charged lipophilic API having a partition coefficient (log P) of more than 0.

More specifically, the invention is applicable to any lipophilic API with log P in the range between 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, or more.

Further, in numerous embodiments the cosmetic lipophilic active ingredient(s) can be dissolved in a cosmetic oil or a mixture of cosmetic oils.

In other embodiments the cosmetic lipophilic active ingredient(s) can be comprised in or comprise a cosmetic oil or a mixture of cosmetic oils.

In these applications, the term “cosmetic oil” implies that it is a carrier or an excipient or inactive substance of lipophilic nature that serves as the vehicle or medium for the cosmetic lipophilic active ingredient.

On the other hand, in numerous embodiments the cosmetic lipophilic active ingredient per se can be a cosmetic oil, meaning that the two terms are partially overlapping.

More broadly, the term “cosmetic oil” encompasses herein any type of oil or cocktail of oils used for cosmetic purposes as a carrier or as an active ingredient to provide nourishment, moisturization and/or more advanced properties such as antiaging, sun protection, anti-wrinkling, etc.

It further encompasses natural and synthetic oils. Examples of such oils are mineral oils and waxes, which are relatively cheap and have good moisturizing properties, and so are used as excipients in many cosmetic products. Frequently used mineral fats are cera microcristallina, ceresin, hydrogenated polyisobutene, isododecane, isohexadecane, ozokerite, paraffin, paraffinum liquidum, petrolatum, synthetic wax. Silicones can be used for their texture in hair care products.

Cosmetic oils further encompass vegetable oils, the natural alternatives for mineral and synthetic fats, which can be extracted from a variety of sources such as fruit, nuts, beans, seeds, cereals, etc. Vegetable oils are known for their moisturizing, nourishing and emollient actions action due to richness in essential fatty acids, vitamins, antioxidants, polyphenols and sterols.

Animal fats are another source of cosmetic oils. This is a relatively cheap material that provides moisturizing, humectant and emollient properties.

Thus, in numerous embodiments the cosmetic oils can be obtained from a vegetable or an animal source, a synthetic oil or fat, or a mixture thereof

In terms of natural oils, this term further encompasses modified and unmodified natural fat or oils. One important example of an unmodified natural oil is castor oil as the base for lipsticks. Other unmodified oils include, but not limited to, almond oil, apricot kernel oil, sesame oil, safflower oil, wheat germ oil, avocado oil, turtle oil and mink oil. Cocoa butter is used to some extent in suntan products.

In further embodiments, the cosmetic oils can be lycopene oil and/or hemp oil, in combination or alone, in various amounts and proportions. Lycopene is considered a powerful antioxidant with many benefits, including sun protection. Hemp oil can play a crucial role in skin health and anti-aging. Incorporation of lycopene and hemp oil in the present compositions have been presently exemplified.

The term “cosmetic oil” further encompasses reconstituted or modified oils. Reconstituted fractionated coconut oil is widely used. Polyglycerol esters of fatty acids are increasing in importance. Hydrogenation has produced stable oils that are useful in cosmetics. Alkyl esters and monoglycerol esters of fatty acids offer a wider range of properties than the original oils. Improvements in the naturally occurring fats and oils have made it possible for them to compete with the natural products.

In numerous embodiments the cosmetic oils can be natural oils, synthetic oils, modified natural oils, or combinations thereof.

In numerous embodiments the cosmetic oils can be selected from acylglycerols, mono-(MAG), di-(DAG) and triacylglycerols (TAG), medium-chain triglycerides (MCT), long chain triglycerides (LCT), saturated or unsaturated fatty acids.

This term also encompasses essential oils which are widely used in cosmetic products as they offer a variety of cosmetic benefits, in addition to their pleasant aroma. Under essential oils is meant herein complex mixtures and extracts and also their isolated compounds. Certain examples of such oils are Cedarwood Organic Essential Oil, Vetiver Essential Oil, Argan Oil, Neroli Essential Oil, Geranium Essential Oil, The Essentials Marula Oil, Grapefruit Essential Oil and blend of oils such as Thieves Essential Oil, etc.

In certain embodiments the cosmetic oil can be more or more essential oils.

A non-limiting list of natural and essential oils that are applicable to the invention is provided in ANNEX A.

Cosmetic oils can be also produced by mixing inert or base oils with specific lipophilic substances, thereby enriching the base oils to obtain specific properties.

In terms of consistencies, this term encompasses oils used in lotions, suspension, creams, gels, emulsions, sticks and powders. In other words, it encompasses cosmetic oils in liquid, semi-solid and solid forms.

In numerous embodiments the cosmetic oils can be solid, semi-solid and/or liquid at room temperature.

Thus, the term “cosmetic oil” herein is all inclusive with regard to oils serving as a basis for cosmetic products for skin, hair and nails, personal care (e.g., soaps and shampoos), skin care, make-up, fragrance, and others. The main categories of cosmetic products according to the FDA are listed in ANNEX A.

In numerous embodiments, the cosmetic oils can be either naturally or artificially enriched in one or more cosmetic lipophilic active ingredient. Specific examples of such oils are oils comprising phytochemicals, vitamins, antioxidants, phenolic oils, aromatic oils, essential oils, extracts of animals or plants, or combinations thereof.

The term “phytochemical” herein is an umbrella term that encompasses various biologically active compounds found in plants that are broadly characterized as having extra cosmetic benefits in addition to the basic. A few examples of well-known phytochemicals are flavonoids, phenolic acids, isoflavones, curcumin, isothiocyanates, and carotenoids.

The term “vitamin” herein broadly refers to an organic molecule that is usually provided in small quantities. Lipophilicity is a substantial problem with many important vitamins, such as vitamins A, D, E and K, all of which have proven benefits to the skin.

The term “antioxidant” herein refers to any compound or combination of compounds that prevent oxidative stress. Notable examples of lipophilic antioxidants with cosmetic benefits are tocopherols, flavonoids and carotenoids.

The term “phenolic oil” is a broad name for oils with relatively high content of phenolic compounds, i.e., oleuropein, hydroxytyrosol and tyrosol. A notable example is olive oil. Essential oils can be also enriched in phenolic compounds.

The term “aromatic oil” broadly refers to any substance, natural, synthetic, reconstituted or modified which exudes aroma. Some of these substances are also aromatic compounds by chemical definition. Limonene is an example of a fragrant compound which is not aromatic by chemical definition. Essential oils are examples of aromatic oils.

The terms “plant and animal extracts”, some of the candidates from the group of plants have been mentioned above. In general, animal extracts or isolated substances are quite common in cosmetic products, notable examples are gelatin, collagen, lanolin, squalene and ambergris, and others. Additional candidates can include extracts of marine animals, types of mussels and marine phytoplankton.

Thus, it should be appreciated that the candidate cosmetic oils and cosmetic lipophilic active ingredients can belong to more than one of the above groups.

In some embodiments, the cosmetic oils can be fragrances or aroma substances.

Traditional perfume is a solution of fragrance compounds dissolved in selected solvents. Alcohol-based perfumes (e.g., eau de parfum, eau de toilette, eau de cologne, au fraiche) are the most common type of fragrance products. There are also alcohol-free fragrance products, mainly in oil or solid forms. The most used oils are jojoba oil or fractionated coconut oil. The essential advantage of oil-based perfumes is that they do not dry out the skin like alcohol solutions, but rather moisturize the skin and improve the fragrance longevity, while the body temperature allows the scent to be released gradually. The fragrance concentration in oil products is quite high, usually around 20%.

Another type of perfume in which fatty substances play an essential role as aroma carriers are solid perfumes. They occur in the form of emulsions, gels and pomades. Various types of waxes are responsible for their consistency. Solid perfumes are provided in the form of a stick or mass pressed into a thimble, as opposed to sprayable perfumes, they are applied to the skin by rubbing. They tend to deliver richer, deeper notes which are released slowly.

In addition, there are popular ‘home scents’ with a variety of diffusing products, e.g., oil burners, atomizers, diffusers and aerosols distributing aroma in the form of a mist.

Nanoemulsions can also act as a matrix for aroma compounds. Alcohol-free perfumes in the form of nanoemulsions (O/W) can contain 5-15% (w/w) of fragrance composition, without additional solubilizers and co-surfactants. Fragrance-loaded nanoemulsions can be stabilized using nonionic surfactants (ethoxylated castor oil or esters of polyglycerol-4 and sebacic and lauryl acids), in the range of 5-10% (w/w). Additionally, up to 1% wt. of natural preservatives (sodium anisate and sodium levulinate) can be used to obtain microbiologically stable products.

Thus, in certain embodiments, the formulations of the invention can serve as a basis for parfum or fragrance products, and specifically solid parfums.

From another point of view, the compositions of the invention can be seen as a composite matter comprising a plurality of micrometric particles each comprising a plurality of lipophilic nanospheres with an average size in the range of about 50 nm to about 900 nm, the at least one cosmetic lipophilic substance is contained in the micrometric particles and is distributed inside and/or outside the lipophilic nanospheres at predetermined proportions, thereby providing improved topical and/or dermal delivery of the at least one cosmetic lipophilic active ingredient.

In other words, the compositions of the invention are a solid particulate matter comprising particles at a micrometric scale, or particles with an average size in a range of between about 10-900 μm, or more specifically with an average size in the range of 10-100 μm, 100-200 μm, 200-300 μm, 300-400 μm, 400-500 μm, 500-600 μm, 600-700 μm, 700-800 μm and 800-900 μm.

In certain embodiments the powders of the invention can comprise particles with an average size in a range of between about 10 μm and to about 300 μm, or more specifically with an average size in the range of 10-50 μm, 50-100 μm, 100-150 μm, 150-200 μm and 250-300 μm.

The micrometric particles of the compositions of the invention, in themselves, are a composite matter comprising lipophilic nanospheres with an average size between about 50-900 nm, and more specifically, an average size in a range between about 50-100 nm, 100-150 nm, 150-200 nm, 200-250 nm, 250-300 nm, 300-350 nm, 350-400 nm, 400-450 nm, 450-500 nm, 500-550 nm, 550-600 nm, 650-700 nm, 700-750 nm, 750-800 nm, 800-850 nm, 850-900 nm and 900-1000 nm (herein an average size is an average diameter).

The size or diameter of the lipophilic nanospheres can be measured by DLS (dynamic light scattering) upon reconstitution of the powder composition in water, such measurements have been presently exemplified.

In numerous embodiments the size of the micrometric particles correlates to the size of the lipophilic nanospheres, meaning that the size of the lipophilic nanospheres governs the size of the of the micrometric particles.

The above implies that the lipophilic nanospheres are essentially entrapped in the micrometric particles. It further implies that this composite matter has certain porosity or arrangement permitting to contain the nanospheres. These two features have been presently exemplified. They are further reflected in the loading and the encapsulation capacity characteristic of the compositions of the invention (see below)

An important feature of the invention is that the shape and size of the lipophilic nanospheres are substantially maintained upon dispersion in water. In other words, due to particular composition and structure of the composite matter, the average size of the nanospheres remains unchanged under various conditions such as lyophilization, long-term storage, fixation and release from matrixes or films such as PVA, etc. The term “substantially maintained” herein implies a deviation of 1-5%, 5-10%, 10-15%, 15-20% or up to 25% in average diameter before and after the manipulation or exposure to certain conditions.

An important feature of the present compositions resides in the distribution of the cometic lipophilic substances, the active ingredients and/or oils, inside and outside the lipophilic nanospheres. This feature is responsible for the properties of immediate and/or prolonged delivery or of release of actives characteristic of the present compositions.

In numerous embodiments the cometic lipophilic active ingredients and/or oils can be distributed inside or outside the lipophilic nanospheres at a ratio of between about 1:0 to 9:1, respectively.

In certain embodiments the cometic lipophilic active ingredients and/or oils can be distributed inside or outside the lipophilic nanospheres at a ratio of between about 4:1, 7:3, 3:2, respectively, meaning that they are present in an excess inside the lipophilic nanospheres.

In other embodiments the cometic lipophilic active ingredients and/or oils can be distributed inside or outside the lipophilic nanospheres at a ratio of between about 3:7 or 1:4, respectively, meaning that they are present in an excess outside the lipophilic nanospheres.

In still other embodiments the cometic lipophilic active ingredients and/or oils can be distributed inside or outside the lipophilic nanospheres at the ratio of about 1:1, meaning that they are present in approximately equal proportions inside and outside the lipophilic nanospheres.

The same feature can be further articulated in terms of encapsulation capacity of the cosmetic lipophilic active ingredients and/or oils into the compositions. The term “encapsulation capacity” refers to the amount or a proportion of the cosmetic lipophilic active ingredients and/or oils entrapped inside the particulate matter, or the powder composition as a whole.

In numerous embodiments the compositions of the invention can have an encapsulation capacity of the cosmetic lipophilic active ingredients and/or oils up to at least about 80% (w/w) relative to the total weigh, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the total weigh.

In other embodiments this feature can be further expressed as an encapsulation capacity of the cosmetic lipophilic active ingredients and/or oils up to at least about 80% (w/w) relative to the initial weight of the old component, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the initial weight of the old component.

This feature is further related to loading capacity the cosmetic lipophilic active ingredients and/or oils onto the compositions. The term “loading capacity” refers to the amount or a proportion of the cosmetic lipophilic active ingredients and/or oils that are loaded onto the powder composition.

In numerous embodiments the compositions of the invention can have a loading capacity of the cosmetic lipophilic active ingredients and/or oils up to at least about 80% (w/w) relative to the total weight, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the total weight.

In other embodiments this feature can be further expressed as a loading capacity of the cosmetic lipophilic active ingredients and/or oils up to at least about 80% (w/w) relative to the initial weight of the old component, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the initial weight of the old component.

Another important feature characteristic of the present compositions is long-term stability or an extended shelf-life. This feature encompasses herein structural, chemical, and functional stabilities. In this instance, the structural stability is reflected in the ability to preserve particle size of the nanospheres upon reconstitution in water. The chemical stability reflects protection against degradation and oxidation under temperature, light and acidic pH, for example. The functional stability is reflected in preservation of properties of immediate and prolonged actives release.

In numerous embodiments the compositions of the invention can have a long-term stability of about at least about 2 years at room temperature, or more specifically up to at least about 2, 3, 4, 5, 6, 7, 8, 9, and 10 years, and more at room temperature.

With respect to core components, in general, the compositions of the invention comprise at least one sugar, at least one polysaccharide and at least one surfactant and at cosmetic lipophilic active and/or cometic oil.

In numerous embodiments the sugars that are applicable to the present compositions can be broadly characterized as short chain carbohydrates and sugar alcohols, and more specifically oligo-, di-, monosaccharides and polyols. Sugars are safe and are ubiquitously used the cometic industry. The sugars can be from natural sources or synthetic.

In numerous embodiments the sugars can be selected from trehalose, sucrose, mannitol, lactitol and lactose.

In numerous embodiments the sugars can be xylitol, sorbitol, maltitol.

The relevant polysaccharides can be broadly characterized as polysaccharides suitable for use in cosmetic industry and generally considered as safe. They can be natural and/or synthetic polysaccharides. Specific examples of natural polysaccharides are fructans found in many grains and galactans found in vegetables, and further methyl-, carboxymethyl- and hydroxypropyl methyl-celluloses, and also pectin, starch, alginate, carrageenan, and xanthan gum. A nonlimiting list of relevant polysaccharides is provided in ANNEX A.

In numerous embodiments the polysaccharides can be selected from maltodextrin and carboxymethyl cellulose (CMC).

The relevant surfactants can be broadly characterized as non-toxic surfactants, including nonionic and anionic surfactants. Examples of anionic surfactants include
(a) carboxylates: alkyl carboxylates-fatty acid salts; carboxylate fluoro surfactants,
(b) sulfates: alkyl sulfates (e.g., sodium lauryl sulfate); alkyl ether sulfates (e.g., sodium laureth sulfate), (c) sulfonates: docusates (e.g., dioctyl sodium sulfosuccinate); alkyl benzene sulfonates, (d) phosphate esters: alkyl aryl ether phosphates; alkyl ether phosphates. Sodium lauryl sulphate BP (a mixture of sodium alkyl sulfates, mainly sodium dodecyl sulfate, C12H25SO4-Na+). The non-ionic surfactant can include polyol esters, polyoxyethylene esters, poloxamers. Polyol esters include glycol and glycerol esters and sorbitan derivatives. Fatty acid esters of sorbitan (Spans) and their ethoxylated derivatives (Tweens, e.g., Tween 20 or 80) are commonly used non-ionic surfactants.

The surfactants can be further selected from the list of cellulose ethers and derivatives, citric acid esters of mono- and diglycerides of fatty acids (CITREM), diacetyl tartaric acid ester of mono- and diglycerides. Additional examples of surfactants used in cosmetic industry are polyoxyethylene (100) stearyl ether (Brij), polysorbates 20 and 80, and lecithin. A nonlimiting list of relevant surfactants (or emulsifiers) is provided in ANNEX A.

In numerous embodiments the surfactants can be selected from ammonium glycyrrhizinate, pluronic F-127 and pluronic F-68.

In other embodiments the surfactants can be selected from monoglycerides, diglycerines, glycolipids, lecithins, fatty alcohols, fatty acids or mixtures thereof.

Yet in other embodiments the surfactants can be sucrose fatty acid esters (sugar esters).

In numerous embodiments the compositions of the invention can comprise any combination of the above component, with more than one agent from the above groups. in various concentrations and proportions.

More generally, in numerous embodiments the cosmetic lipophilic active ingredients and/or oils can constitute between about 10% to about 98% of the compositions of the invention (w/w), or more specifically between about 10%-20%, 20%-30%, 30%-40%, 40%-50%, 50%-60%, 60%-70%, 70%-80%, 80%-90% and 90%-98% of the present compositions (w/w), or up to about 10%, 20%, 30%, 40%, 50%, 60%, 70%, 80%, 90% and 98% of the present compositions (w/w).

Further, in numerous embodiments the present compositions can further comprise cosmetic carriers, cosmetic excipients, and other additives for purposes of color, taste, and specific consistencies. The terms ‘carriers and excipients’ encompass herein any inactive substances that serve as the vehicle or medium for the cometic lipophilic active ingredients and oils comprised in the compositions. In cosmetic products, widely used excipients also include vegetable oils, mineral oils or synthetic oils (silicones).

On the subject of biological effects, the distribution of the lipophilic active ingredients and oils inside and outside the nanospheres has direct relevance to the feature of improved topical and/or dermal delivery of the lipophilic active ingredients characteristic of the present compositions.

In numerous embodiments the improved topical and/or dermal delivery of the cosmetic lipophilic active ingredients and cosmetic oils comprises an immediate and/or a prolonged topical and/or dermal delivery of the cosmetic lipophilic active ingredients and cosmetic oils.

In numerous embodiments the improved topical and/or dermal delivery of the cosmetic lipophilic active ingredients and cosmetic oils comprises an improved topical and dermal bioavailability of the cosmetic lipophilic active ingredients and cometic oils.

In numerous embodiments the improved topical and/or dermal delivery of the cosmetic lipophilic active ingredients and cosmetic oils comprises an improved release of the cosmetic lipophilic active ingredients and cometic oils into at least one layer of the skin.

In numerous embodiments the improved topical and/or dermal delivery of the cosmetic lipophilic active ingredients and cosmetic oils comprises an improved penetration and/or permeation of the cosmetic lipophilic active ingredients and cometic oils through at least one layer of the skin.

Skin and dermatological permeability is different from the oral bioavailability. Chemicals and bioactives are transported through the skin predominantly by three main mechanisms: (1) penetration of a substance via the skin layers and accumulation in a particular layer of stratum corneum; (2) permeation of a substance through one layer into another via hydrophilic channels in the dermis (such as hair roots); (3) resorption is the uptake of a substance into the vascular system (lymph and/or blood vessel) in the dermis and hypodermis. In the non-therapeutic context, resorption should be avoided.

The terms “penetration” and “permeation” in the context of the skin encompasses herein topical and dermal absorption as the total sum of the two processes. In cosmetics, resorption or penetration of actives to the circulation should be avoided.

The notion of improved skin delivery has been based on the finding that the formulations retain their essential structure and particle size under the conditions of high osmolarity mimicking human skin (EXAMPLE 1.8) together with findings of improved release of actives and permeation into the skin (EXAMPLE 6).

The term “improved” encompasses herein a change in a range of about 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, 95-100% relative to oil forms with the same actives, or up to 2, 3, 4, 5, 6, 7, 8, 9, 10, 20, 30, 40, 50, 60, 70, 80, 90, 100 fold relative to oil forms with the same actives.

This term further encompasses any advantageous change in the availability pattern, and specifically changes related to modulation of availability, such as those revealed in the present compositions.

More specifically, in certain embodiments the compositions of the invention can provide an immediate topical and dermal delivery of cosmetic lipophilic active ingredients and oils into one or more layers of the skin.

The term “immediate delivery” in the context of skin implies a topical and dermal delivery of active within a relatively short period of time, such as up to 1, 10, 20, 30, 40, 50, 60, 70, 80, 90, 100, 110, 120 min or more from the onset of topical administration.

In other embodiments the compositions of the invention can provide a prolonged topical and dermal delivery of cosmetic lipophilic active ingredients and oils into one or more layers of the skin.

The term “prolonged delivery” in the context of skin implies a topical and dermal delivery of active within a longer period, such as up to 30, 60, 90, 120, 150, 180, 210, 240, 270, 300, 330, 360 min (6 h) and further up to 7, 8, 9, 10, 11, 12 h or more from the onset of topical administration.

Yet in other embodiments the compositions of the invention can provide a biphasic actives release profile comprising an immediate and a prolonged delivery of cosmetic lipophilic active ingredients and oils into one or more layers of the skin.

Modulation of actives release and bioavailability can have significant impact on the considerations of effective dosing and the final concentrations of active included in the cosmetic product to achieve the desired outcome, e.g., better appearance of the hair and/or skin, and subject's satisfaction with the product.

All the above further apply to additional important aspects of the invention, including dosage forms, cosmetic products, and other applications.

More specifically, it is another objective of the invention to provide a dosage form comprising an effective amount of the compositions according to the above.

On the whole, in numerous embodiments the compositions and dosage forms of the invention are provided in a form or are adapted for topical and dermal administrations.

The concept of dosage form is attractive as it implies carefully controlled amounts of actives comprised in the formulations of the invention, the cosmetic oils and/or other bioactives as above.

The term “effective amount” broadly relates to an amount of active needed to provide a desired level cosmetic effect in the sense of the definition by FDA (cleansing, beautifying or altering the appearance without affecting the body's structure or functions). In many instances such effects are subjective and the decision on the effective dose is made based on customers compliance and marketing strategy.

This term further implies loading of the cosmetic lipophilic actives and oils into the compositions or dosage forms. The present compositions have been demonstrated as particularly capable of high loading and encapsulation of cosmetic actives and oils.

Thus, in numerous embodiments the compositions and dosage forms can include cosmetic lipophilic active ingredients and oils at the proportion in the range of about
0.01%-0.1%, 0.01%-1% (w/w) of the total weight, and further in the range of about
1-5%, 5-10%, 10-15%, 15-20%, 20-25%, 25-30%, 30-35%, 35-40%, 40-45%, 45-50%, 50-55%, 55-60%, 60-65%, 65-70%, 70%-75%, 80%-85%, 85%-90%, 90%-95% and 95%-100% (w/w) of the total weight, depending on the other desired cosmetic effects and other properties such as viscosity, texture, translucency, etc.

In yet another aspect, the invention can provide a kit comprising the above compositions and/or dosage forms. The concept of a “kit” implies a cosmetic product provided in portions for single or multiple repeated uses. It further implies a package, a capsule or a container containing the separate portions, and instructions for use.

A specific example of a kit or a dosage form is a dermal patch, wherein the compositions are embedded in a plasticized material in carefully controlled doses. Patches using PVA have been presently exemplified (EXAMPLE 5).

Alternative plasticizing materials can include but, are not limited to, synthetic resins such as polyvinyl acetate (PVAc) and sucrose esters and natural resins such as rosin esters (or ester gums), natural resins such as glycerol esters of partially hydrogenated rosins, glycerol esters of polymerised rosins, glycerol esters of partially dimerised rosins, glycerol esters of tally oil rosins, pentaerythritol esters of partially hydrogenated rosins, methyl esters of rosins, partially hydrogenated methyl esters of rosins and pentaerythritol esters of rosins. Also, synthetic resins such as terpene resins derived from alpha-pinene, beta-pinene, and/or d-limonene and natural terpene resins.

Thus, in certain embodiments the dosage forms of the invention can be provided in a form of a dermal patch.

More generally, the compositions of the invention can serve as a basis for a wide range of cosmetic products. Thus, the invention can be further articulated as a cosmetic material comprising the formulations or dosage forms of the invention.

The term “cosmetic material” herein is an umbrella term encompassing a wide range of raw materials used for manufacturing of cosmetic products, e.g., oily materials such as oils, fats, wax esters, and ester oils; humectants such as polysaccharides, proteins, acids and small molecules like aloe vera, glycerin, sorbitol and others.

It should be remembered that the compositions of the invention are compatible with water-based materials.

In numerous embodiments the cosmetic material can be in the form of a powder, a stick, a lotion, a gel, a cream, an emulsion, a patch.

In certain embodiments the invention can provide a non-alcoholic parfum material comprising the compositions and dosage forms according to the above.

In further embodiments the non-alcoholic parfum material can be in the form of a powder, a stick, a lotion, a cream, an emulsion, a liquid, an essence, a patch.

In other embodiments the invention can provide a solid non-alcoholic parfum comprising the compositions and dosage forms according to the above.

Yet in other embodiments the invention can provide a sunscreen material comprising the compositions and dosage forms according to the above.

In further embodiments the sunscreen material can be in the form of a stick, a lotion, a gel, a cream, an emulsion.

Still in other embodiments the invention can provide a haircare material comprising the compositions and dosage forms according to the above.

In further embodiments the haircare material can be in the form of a liquid or solid soap, a liquid or solid shampoo, an emulsion, a gel, a lotion, a cream.

In other embodiments the invention can provide a personal care material comprising the compositions and dosage forms according to the above.

In further embodiments the personal care material can be in the form of a liquid or solid soap, an emulsion, a lotion, a gel, a cream.

In other embodiments the invention can provide a make-up material comprising the compositions and dosage forms according to the above.

In further embodiments the make-up material can be in a solid or a liquid form.

In other embodiments the invention can provide a skin care material comprising the compositions and dosage forms according to the above.

In further embodiments the skin care material can be in a form of a powder, a lotion, a cream, an emulsion, a gel.

Still in other embodiments the invention can provide a solid shampoo comprising the compositions and dosage forms according to the above.

A non-limiting list of cosmetic products that can be made from these cosmetic materials is provided in ANNEX A.

Yet from another point of view, the invention can be articulated in terms of compositions and dosage form according to the above that can be used in improving topical or dermal delivery of one or more cosmetic lipophilic active ingredients and cosmetic oils.

In numerous embodiments, the compositions and dosage forms of the invention can be used for UV protection, skin moisturizing, skin whitening, skin tanning, skin anti-wrinkling, skin elasticity promoting, skin antiaging agent, skin exfoliation, or any combination thereof.

Still from another aspect, the invention can be articulated in terms of use of the present compositions in the manufacture of a cosmetic material with improved topical or dermal delivery of cosmetic lipophilic active ingredients and cosmetic oils.

It is another objective of the invention to provide a series of methods for improved delivery of cometic actives and oils, and non-therapeutic cosmetic applications thereof.

In numerous embodiments these methods can be articulated in terms of
non-therapeutic methods for cosmetic treatment of a subject, comprising topical administering to the subject an effective amount of the compositions and dosage forms according to the above.

More specifically, according to the invention, the non-therapeutic method for cosmetic treatment of a subject comprise topical administering to the subject an effective amount of a solid water-dispersible composition of matter comprising at least one sugar, at least one polysaccharide and at least one surfactant and at least one cosmetic lipophilic active ingredient, and wherein the composition comprises a plurality of micrometric particles each comprising a plurality of lipophilic nanospheres with an average size in the range of about 50 nm to about 900 nm, the at least one cosmetic lipophilic substance is contained in the micrometric particles and is distributed inside and/or outside the lipophilic nanospheres at predetermined proportions, thereby providing an improved topical and/or dermal delivery of the at least one cosmetic lipophilic active ingredient to the subject.

In numerous embodiments said improved topical and/or dermal delivery of cosmetic lipophilic active ingredients the subject can comprise an immediate and/or a prolonged topical and/or dermal delivery of the cosmetic lipophilic active ingredients to the subject.

In numerous embodiments said improved topical and/or dermal delivery of the cosmetic lipophilic active ingredients to the subject can comprise an improved topical and/or dermal bioavailability of the cosmetic lipophilic active ingredients in the subject.

Still in numerous embodiments said improved topical and/or dermal delivery of the cosmetic lipophilic active ingredients to the subject can comprise an improved penetration and/or permeation of cosmetic lipophilic active ingredients through at least one layer of the subject's skin.

In numerous embodiments said improved topical and/or dermal delivery of the cosmetic lipophilic active ingredients to the subject can comprise an improved release of the cosmetic lipophilic active ingredients into at least one layer of the subject's skin.

Still from another point of view, the invention provides methods for improving topical and dermal delivery of cosmetic lipophilic substances, cosmetic lipophilic active ingredients and oils, involving topical administering to a subject an effective amount of the formulations and dosage forms according to the above.

As has been noted, in numerous embodiments, such improved delivery of lipophilic substances comprises immediate and prolonged penetration and/or permeation of the cosmetic lipophilic substances into the skin, or both. Additional embodiments of such application have been previously discussed.

Ultimately, the invention provides methods of making compositions with an improved topical and/or dermal delivery of one or more cosmetic lipophilic active ingredients and cosmetic oils, with the general steps of:
mixing an aqueous phase comprising at least one sugar, at least one polysaccharide and at least one surfactant with an oil phase comprising at least one cosmetic lipophilic active ingredient,
b. (ii) emulsifying the mix to obtain a nanoemulsion,
c. (iii) lyophilizing or spray dying the nanoemulsion.

The invention further provides methods of making compositions with an increased loading of cosmetic lipophilic active ingredients and cosmetic oils, with the general steps of:
mixing an aqueous phase comprising at least one sugar, at least one polysaccharide and at least one surfactant with an oil phase comprising at least one lipophilic API,
d. (ii) emulsifying the mix to obtain a nanoemulsion,
e. (iii) lyophilizing or spray dying the nanoemulsion.

The term “about” in all its appearances in the text denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

Any methods and materials similar or equivalent to those described herein can be used in the practice or testing of the present invention. Some embodiments of the invention will be now described by way of examples with reference to respective figures.

### Example 1: Physical Properties of the Powder Formulations

### 1.1 Preservation of Particle Size in the Reconstituted Powder Compositions

A powder composition comprising 30% of AlaskaOmega (Omega 3) was prepared by nano-emulsification, freezing in liquid N2 and lyophilization (48 h). Distribution and uniformity of the particle size was evaluated after nano-emulsification and lyophilization upon dispersion of the powder in TWD to 1% (w/w) using PDI (poly dispersity index) measured by DLS (dynamic light scattering). Measurements were perfumed in triplicates. PDI correlates to particle size.

PDI values indicated that the nanoemulsion and the reconstituted powder contained a uniform and homogenous population of particles with the average size of
149 nm±SD and 190 nm±SD, respectively.

Overall, the results suggest that the particle size in the nanoemulsions and the respective reconstituted powder compositions is relatively constant, and that this feature is relatively uniform and homogeneous per sample, overall. The feature of preservation of particle size upon reconstitution in water characteristic of the compositions of the invention applies to water environments, in general, and to circulation, tissues and the GI in particular.

1.2 Preservation of Particle Size after Storage for 1 Month

Powders stored for 1 month were reconstituted in TWD to 1% (w/w) or 2% (w/w) and subjected to DLS or Cryo-TEM (transmission electron cryo-microscopy) analyses. The average particle size in the reconstituted powders was 218 nm±SD and 100 nm±SD for DLS and Cryo-TEM, respectively, suggesting certain differences in measurements due to technology.

Overall, the results suggest that the powder compositions of the invention are relatively stable in retaining the reconstitution capacity and uniform population of particles at a nanometric range.

1.3 Powder Compositions with Lycopene Oil and Hemp Oil

Powder compositions were produced from a combination of lycopene oil and hemp (1:1.4, respectively) using the above method. DLS analysis was performed on the nanoemulsion and the reconstituted powder (1% w/w).

DLS analysis showed a single population of particles in the nanoemulsion with the average size of 590 nm and two populations of particles in the reconstituted powder with the average size of 272 nm and a minor peak at 79 nm. The particle size was not increased after lyophilization.

The results suggest that in terms of preservation and uniformity of particle size, the powder compositions with lycopene and hemp oil behave similarly to the powders with Omega 3. Overall, the results suggest that the technology is adaptable to various types of lipophilic carriers, i.e., oils and combinations of oils.

1.4 Preliminary Stability Studies with Compositions Comprising Cannabinoids

Powder compositions with CBD or THC were stored at 45° C. (oven) for 1, 35, 54, 72 and 82 days (3 months correlates to 24 months at RT). Particle size was evaluated using DLS. The results are shown in Table 1 and FIG. 1.

TABLE 1

| DS measurements in test samples¶ | | | | | |
|---|---|---|---|---|---|
| Temp | | AVG | PDI | PEAK | ¤ |
| RT | | 150.5 | 0.208 | 163.2 | ¤ |
| 1 day at | 45° C.□ | 149.1 | 0.213 | 151.9 | ¤ |
| 35 days at | 45° C.□ | 160.2 | 0.25 | 159.6 | ¤ |
| 54 days at | 45° C.□ | 150.1 | 0.216 | 144.7 | ¤ |
| 72 days at | 45° C.□ | 150.1 | 0.212 | 143.3 | ¤ |
| 82 days at | 45° C.□ | 153.7 | 0.205 | 154 | ¤ |

AVG average diameter (nm)¶
PDI polydispersity index¶

The results show that the particle size was preserved for at least three months at 45° C., thus providing additional evidence for long-term stability and ability to preserve particle size under various conditions characteristic of the present powder compositions.

1.5 Compositions with Lactose and Hemp Oil

Nanoemulsions and the respective powders were prepared with lactose as a choice of sugar. The list of ingredients is detailed in Table 2.

TABLE 2
Specifications of test samples¶

| Lactose• | 80%¤ | 90%¤ | 100%¤ | 110%¤ | 120%¤ |
|---|---|---|---|---|---|
| Ammonium Gly¤ | 3.05¤ | 3.05¤ | 3.05¤ | 3.05¤ | 3.05¤ |
| Meltodextrin¤ | 13.68¤ | 13.68¤ | 13.68¤ | 13.68¤ | 13.68¤ |
| Lactose¤ | 16¤ | 18¤ | 20¤ | 22¤ | 24¤ |
| Water¤ | 145.74¤ | 145.74¤ | 145.74¤ | 145.74¤ | 145.74¤ |
| Hemp oil¤ | 15.74¤ | 15.74¤ | 15.74¤ | 15.74¤ | 15.74¤ |

Nanoemulsions were prepared from lactose solution (80%) and maltodextrin (25-50° C.), lactose was added to the concertation of 90%, 100%, 110%, 120% (relative to the initial concentration), and then Ammonium Gly and hemp oil. The nanoemulsions were homogenized by M-110EH-30 at 10,000-20,000 PSI (25-50° C.)×4 cycles. Powders were prepared by (1) lyophilization, i.e., freezing (−25° C. to −86° C.) and lyophilization (12-24 h, −51° C., 7.7 mbar); or (2) spray drying using peristaltic pump (rate 8.5-20 g/min, air temp. 110-150° C., air flow 0.4-0.5 m3/min, atomizer pressure 0.15 MPa). DLS analysis of the reconstituted powders is shown in Table 3.

TABLE 3
DS measurements in tested samples

| Lactose | Drying | Yield | | Pump rate | Average Size |
|---|---|---|---|---|---|
| conc. | technology | (%) | T air out | (g/min) | (nm) |
| 80% | Spray dryer | 54.8 | 62 | 8.78 | 135.3 |
| 90% | Spray dryer | 63.8 | 62 | 9.66 | 127.6 |
| 100% | Spray dryer | 87.5 | 63 | 10.4 | 125.6 |
| 120% | Spray dryer | 87 | 63 | 10.1 | 124.6 |
| 80% | Lyophilizer | 100% | NR | NR | 136.1 |
| 100% | Lyophilizer | 100% | NR | NR | 127.8 |
| 110% | Lyophilizer | 100% | NR | NR | 125.4 |
| 120% | Lyophilizer | 100% | NR | NR | 124.5 |

The results show preservation of particles size with various concentrations of lactose and under various manipulations. Overall, the results suggest that lactose can serve as an alternative sugar without disrupting the core properties of the composition.

### 1.6 Loading Capacity and Distribution of the Lipophilic Component

Nanoemulsions were prepared with various types of oil carriers: Omega 7, TG400300, EE400300. Surface oil content was determined by hexane. Powders (5 g) were washed with hexane (50 ml), filtered, and washed (×4) with hexane (5 ml). Loss on drying (LOD) was performed on the filtrate under N2 until stabilization of weight. The oil content inside the nanospheres was estimated as:
Omega 7-52.67%
TG400300-30.67%
EE400300-35.33%

The results suggest that up to 50% lipophilic carrier can be incorporated into the lipophilic nanospheres, depending on the type of oil. A similar distribution can be assumed for the lipophilic actives dissolved in such carriers. The results further suggest that a substantial proportion of lipophilic carrier (and active) can be present outside the nanospheres. This finding strongly supports the notion of differential bioavailability and biphasic release of lipophilic actives characteristic of the present powder compositions.

More recent studies suggested that above 80% and 90% of lipophilic carriers and actives can be incorporated into the nanospheres.

Overall, these results are indicative of high loading capacity of lipophilic carriers and actives in the powder compositions of the invention.

### 1.7 Encapsulation Capacity of the Compositions

Encapsulation capability was estimated by the difference between the initial amount of the lipophilic carrier/active and the final amount of the carrier/active unentrapped in the composition. Four different types of powders were prepared with the following lipophilic carriers/actives using the above methods:
Vitamin D3 oil
Passionfruit oil
Medium-chain triglyceride (MCT) oil
Pomegranate seed oil

The non-encapsulated lipophilic carriers/actives were removed with hexane (shaking 1 g powder in 10 ml n-Hexane for 2 min), the product was filtered and washed with hexane (×3), and the content of the entrapped carriers/actives was measured using Solvent extraction-gravimetric method. The results are shown in Table 4.

TABLE 4
The entrapped oil content of tested compositions¶

| | Before wash | After wash | Encapsulation |
|---|---|---|---|
| Oil/active¤ | (gr/100 gr)¤ | (gr/100 gr¤ | efficiency¤ |
| Vitamin D¤ | 30.57¤ | 30.50¤ | 99.8%¤ |
| Passion fruit¤ | 30.31¤ | 29.46¤ | 97.2%¤ |
| MCT¤ | 29.06¤ | 28.79¤ | 99.1%¤ |
| Pomegranate¤ | 29.16¤ | 26.11¤ | 99.8%¤ |

The results point to a substantially highly loading capacity of lipophilic carriers/actives into the present compositions up to the extent of 97.0-99.8% of the total lipophilic content.

### 1.8 Preservation of Particle Size in High Osmolarity Solutions

The feature of preservation of particle size was further studied in saline solution mimicking the osmolarity on the human skin. To be compatible with the skin, topical compositions should be stable and maintain their characteristic properties in high salinity solutions-typically 0.5-0.8% NaCl. The nanometric powders were resuspended

(1% w/w) in the saline solution (0.75% NaCl) and in TDW. DLS analysis was performed as above. Tests were performed in triplicates.

The raw data of the distribution of particle size is given below:
Water: Z Avg; 164.1 nm, pdi: 0.232, peak1: 175.3 (99.3%), peak2: 3508 (0.7%).
Saline Sol.: Z AVG; 158.2 nm, pdi: 0.236, peak1: 154.3 (98.6%), peak2: 4085 (1.4%).

The results show minor differences in the particle size between the saline solution and water, 158 nm vs. 164 nm, respectively. The results suggest that the powder compositions can retain their original particle size and uniformity in high osmolarity solutions. Preservation of nanometric particle size and larger surface area can provide deeper penetration of carriers/actives into the skin and improved efficacy.

Overall, the study suggests that the powder compositions can be applicable for topical and dermal delivery of therapeutic and non-therapeutic lipophilic actives.

1.9 Compositions with Additional Lipophilic Carriers

Powders were prepared with various lipophilic carriers using the above methods:
Sample 1—Fish oil FO 1812 Ultra, 50% oil
Sample 2—KD-PUR 490330 TG90 Ultra, 30% oil
Sample 3—KD-PUR 490330 TG90 Ultra, 50% oil

Particle size was evaluated in the nanoemulsions and the reconstituted powders as above. The particle size remained surprisingly stable in the respective nanoemulsions and reconstituted powders, with an average size ranging from about 140-160 nm.

In summary, the different compositions showed consistency of particle size in the transition from nanoemulsion to solid forms. The particles size remained stable during the drying process, which was highly surprising because of extreme conditions of temperature and drying. This experiment suggests wide applicability of the present technology to numerous types of lipophilic carriers and actives.

1.10 Compositions with High Content of Oils and Lipophilic Actives

Several examples of powder compositions with high content of lipophilic carriers/actives are provided below:

Curcumin 70%

| Ingredient | amount (gr) |
|---|---|
| Sucrose | 9.1 |
| Maltodextrin | 6.1 |
| Ammonium Gly | 2.8 |
| Curcumin extract powder | 42 |
| Water | 140 |

Sucrose and Maltodextrin were fully dissolved in water; curcumin powder was dry blended with Ammonium Gly and added to the solution; initial emulsion was produced and fed into the microfluidizer (4 bar, 16,000 PSI, ×2 cycles).

Q10 100%

| Ingredient | amount (gr) |
|---|---|
| Ammonium Gly | 4 |
| Q10 | 56 |
| Water | 140 |

Q10 powder was dry blended with Ammonium Gly, mixed and homogenized with water until homogenous emulsification, and fed into the microfluidizer (4 bar, 16,000 PSI, ×2 cycles).

CBD and MCT 70% oil

| Ingredient | amount (gr) |
|---|---|
| Sucrose | 7.6 |
| Maltodextrin | 5 |
| Ammonium Gly | 2.4 |
| Glycerin | 3 |
| CBD | 21 |
| MCT | 21 |
| Water | 140 |

CBD was dissolved in MCT at 40° C. and Ammonium Gly was added until even dispersion. Sucrose, Maltodextrin and Glycerin were dissolved in the water. The mixture of oil and active was added to, emulsified, and fed into the microfluidizer (4 bar, 16,000 PSI, ×2 cycles).

### Example 2: Surprising Chemical Stability of Actives

2.1 Stability of Compositions with Cannabis Extracts

Cannabinoids are especially prone to chemical and photolytic degradation. Nanoemulsions were prepared with full spectrum Cannabis oil (50%) obtained from two Cannabis strains (THC or CBD enriched) and the other core components of the present compositions. The reconstituted powders yielded the characteristic particle size of about 150 nm and the original cannabinoid spectrum in oil. Powders were stored in aluminum bags in 40° C. chamber under the following conditions:
1 gr per bag
O2 scavenger
Silica humidifier

The experiment was performed in two independent runs for powders with THC and CBD enriched extracts (Powder A and Powder B). Cannabinoid analysis was performed using HPLC at Baseline (0), 30 days, 45 days, and 83 days (correlates to 10, 13, 24 months at RT). The results are shown in Tables 5 and 6.

TABLE 5
Cannabinoid analysis in Powder A

| Analyte content | | | | | Total |
|---|---|---|---|---|---|
| (% w/w) | THC-Δ-9 | CBD | CBG | CBN | cannabinoids |
| T0 | 2.71 | 1.05 | 0.09 | 0.08 | 3.93 |
| 10 months | 2.62 | 1.03 | 0.09 | 0.09 | 3.83 |
| 13 months | 2.68 | 1.02 | 0.07 | 0.09 | 3.86 |
| 24 months | 2.62 | 1.03 | 0.09 | 0.09 | 3.83 |

TABLE 6
Cannabinoid analysis in Powder B

| Analyte content | | | | | Total |
|---|---|---|---|---|---|
| (% w/w) | THC-Δ-9 | CBD | CBG | CBN | cannabinoids |
| T0 | 0.28 | 3.95 | 0.01 | 0.07 | 4.31 |
| 10 months | 0.28 | 3.98 | 0.01 | 0.02 | 4.29 |
| 13 months | 0.27 | 3.93 | 0.01 | 0.09 | 4.3 |
| 24 months | 0.28 | 3.98 | 0.01 | 0.02 | 4.29 |

The results suggests that the compositions of the invention provide long-term stability for actives such as cannabinoids, and complex compositions of cannabinoids (Cannabis extracts) for a minimum of at least 24 months at RT. The recommended storage conditions are in aluminum bags with 02 scavenger and/or moister desiccator.

Overall, under these conditions, the maximum degradation rate did not exceed 2.5% for the entire cannabinoid content and was even lower for specific cannabinoids (THC and CBD as CBN and CBG). The feature of long-term stability was further supported by the content of CBN, as a known marker of cannabinoid degradation.

### 2.2 Stability of Compositions Comprising Lycopene

Carotenoids are known to be sensitive to increased temperature, pro-oxidative species, and acidic pH. Nanoemulsions were prepared with lycopene oleoresin (6% lycopene w/w) and the other core components. Powders (4 gr) were heat-sealed with vacuum in aluminium bags with moister and oxygen scavengers, and stored for 0, 30, and 90 days at RT (25° C.), 4° C. and 40° C. (in duplicates). Products were tested by visual appearance, DLS and HPLC analyses at the indicated time points.

Visual analysis suggested that all samples preserved the typical confluence texture, and color during the storage period. DLS analysis indicated that the original particle size of 225-272 nm was relatively preserved. The results are shown in Table 7. HPLC analysis showed minimal losses of lycopene during the storage period, i.e., 7%, 3%, and 1% for samples stored at RT, 4° C., and 40° C., respectively.

TABLE 7
DLS analysis of compositions with lycopene¶

| Storage temperature¤ | Time 0¤ | Time 30 days¤ | Time 90 days¤ |
|---|---|---|---|
| RT (about 25° C.)¤ | 260 nm¤ | 225 nm¤ | 236 nm¤ |
| 4° C.¤ | | 272 nm¤ | 265 nm¤ |
| 40° C.¤ | | 246 nm¤ | 251 nm¤ |

Overall, the results suggest that the powder compositions can provide an extended shelf life for actives such as lycopene and can prevent their oxidation and degradation. Extended stability of 90 days at 40° C. corresponds to 2-years at RT. The recommended conditions should further include aluminum bags with moister and oxygen scavengers.

2.3 Stability of Compositions with Vitamin D3

Powders comprising vitamin D3 were stored under 40° C./RH 75° C. for 90 days. Vitamin D3 and ethoxy vitamin D3 degradation products were detected by HPLC. Analytical tests were further validated by an external authorized laboratory (Eurofins). The results are shown in Table 8.

TABLE 8
HPLC analysis of compositions with vitamin D3¶

| | | | Vitamin D | | | |
|---|---|---|---|---|---|---|
| ¤ | Vitamin D | | degradation product | | Eurofins results | |
| Vitamin D3 oil¤ | 24.14 | mg/gr¤ | 0.70 | mg/gr¤ | 26.3 | mg/gr¤ |
| Vitamin D3 powder | 6.76 | mg/gr¤ | 0.40 | mg/gr¤ | 7.7 | mg/gr¤ |

| | | | | | |
|---|---|---|---|---|---|
| Day 1¤ | | | | | |
| Vitamin D3 powder | 6.60 | mg/gr¤ | 0.47 | mg/gr¤ | Duplicate•1-6.9•mg/gr¶ |
| Day 90¤ | | | | | Duplicate•2-7.4•mg/gr¤ |

The cholecalciferol tests were consistent with the certificate (1M iu/g). The results indicated that the encapsulated fraction contained 28%-29% vitamin D3 compared to the 30% vitamin D3 in the original oil preparation, suggesting minimal losses of vitamin D3 during the production process. In addition, only minimal degradation was observed during the storage period (up to 5% active). The differences between duplicates can be explained by soldering. The powder had a far fewer degradation products compared to the oil form. Overall, the study suggested potential stability of the powder form for a period of at least 2 years at RT.

The above studies suggest that the powder compositions of the invention have surprisingly long shelf-life and capability to preserve chemical stability of actives. This feature is highly surprising, especially in view that the production process involves high pressure, water environment, both of which are unfavorable for lipophilic molecules, and further in view that the reduction of particle size and the increase in surface area are expected to increase oxidation and chemical instability actives. These findings further support pharmacological applicability of the present compositions and methods, and especially for poorly water-soluble and lipophilic actives.

2.4 Stability of Compositions with Fish Oil

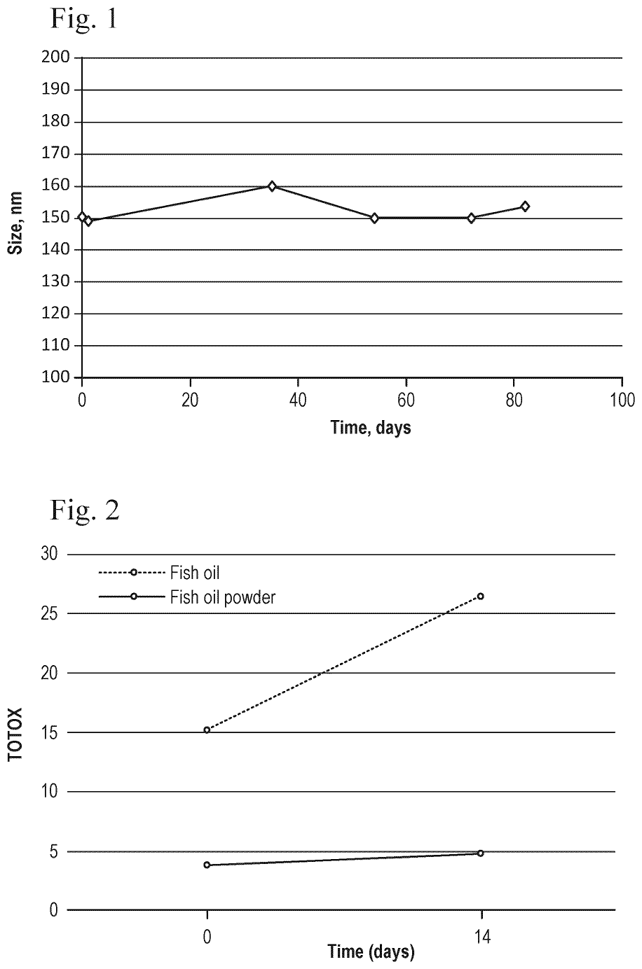

The feature of chemical stability of actives was further supported in a comparative study of fish oil, per se, and fish oil formulated in the powder composition of the invention. Fish oil (60% Omega 3 fatty acids w/w) are known to oxidize readily by forming primary and secondary oxidation products, which can be harmful for humans. Powder compositions were prepared from 40% fish oil (w/w) and the core other components. The powder and oil samples were exposed to environmental oxygen, heat-sealed with vacuum, and stored at 4° C. for 28 days. The primary (peroxide; PV) and secondary (anisidine; AV) oxidation products were measured at days 0, 14, and 28. TOTOX value (overall oxidation state) was calculated using Formula:
TOTOX=AV+2*PV. The results are shown in FIG. 2.

The results show that the powder composition had a significantly lower TOTOX, i.e., a significantly lower concentrations of primary and secondary oxidation products, compared to the oil form starting from day 0 and up to day 14. The result of day 0 is further indicative that the production process of the powders does not lead to degradation, despite the exposure to water and oxygen.

Overall, the results support a surprising capacity of the powder composition to protect actives and prevent their oxidation/degradation, most likely due to encapsulation. This property is further consistent with the previously shown characteristic of long-term stability and extended shelf-life.

### Example 3: Surprising Loading Capacity

Loading capacity of the powder compositions was further studied in the example of concentrated Cannabis oil. Nanoemulsions were produced with raw RSO high THC concentrate (1 gr) by the above methods. The nanoemulsions and the reconstituted powders yielded the characteristic particle size of about 150 nm. The reconstituted powders were subjected to analysis of cannabinoids using HPLC. Table 9 shows the calculated vs. actual cannabinoid content.

TABLE 9
The measured and calculated THC content¶

| % w/w | Calculated | Measured |
|---|---|---|
| Δ9-THC¤ | 8.945%¤ | 8.45%¤ |
| CBG¤ | 0.276%¤ | 0.24%¤ |

The ratio between the calculated and actual content was 94.91%, and 86.9% for Δ9-THC and CBG, respectively, suggesting minimal losses of actives. The proportion of oil carrier relative to the total powder material further suggested a surprisingly high loading capacity of lipophilic carriers and actives in the present powder compositions.

### Example 4: Surprising Profiles of Active Release

4.1 Pharmacokinetic Studies of Compositions with Cannabinoids

Pharmacokinetic profiles (PK) of the compositions of the invention were evaluated in a rat model. The study compared PK profiles in plasma and in selected organs (liver and brain) of two types of compositions: the powder compositions with CBD/THC (LL-P) and the oil compositions with the same active (LL-OIL).

The study used the following end points:
a. Mortality and morbidity monitoring—daily.
b. Body weight monitoring—during acclimation and before dosing.
c. Clinical observation—prior to and for 2 h after oral administration.
d. Blood draws—at timepoints of 0, 15, 30, 45, 60, 90, 120 and 240 min.
e. Termination and organ collection (brain, liver) at 45, 60, 90, 120, 240 min.

The study used classical PK procedures in animals (N=12) divided into 2 groups.

### Materials and Methods

Test item I: CBD/THC POWDER (LL-P): LL-CBD-THC 30% oil in powder
Test item II: CBD/THC OIL (LL-OIL): LL-CBD-THC oil diluted in hemp oil

Oral doses (per animal) were prepared as follows: 225 mg of LL-P was dissolved in 4.275 mg TDW; 67.5 mg LL-OIL was diluted in 1 ml hemp oil.

Male rats/12/376/456 g (sex/number/weight) were divided into groups (deviation of ±20% from mean weight in each group) and acclimatized (8 days). The study (1 cycle) was conducted in 2 groups (×6 animals, ×3-4 time points). Blood samples were collected at indicated time-points and stored. Organs (brain, liver) were collected after terminal bleeding and perfusion, and stored. Group allocations are shown in Table 10. There were no findings of morbidity, pain, or distress during the entire study period.

TABLE 10
Group allocation

| | | Dose | | | | | | |
|---|---|---|---|---|---|---|---|---|
| | Dose | volume | | | | | | |
| Group | (mg/kg) | (ml/kg) | Route | Animal | Bleeding time point | | Termination | |
| LL-P | THC 13.5 | 10 | Oral | 1 | 0, 15, 45 | min | 45 | min |
| | CBD 15.7 | | | 2 | 0, 30, 60, 240 | min | 240 | min |
| | | | | 3 | 15, 45, 60 | min | 60 | min |
| | | | | 4 | 30, 60, 90 | min | 90 | min |
| | | | | 5 | 45, 90, 120 | min | 120 | min |
| | | | | 6 | 0, 15, 30, 90 | min | 90 | min |
| LL-OIL | | 3 | | 7 | 0, 15, 45 | min | 45 | min |
| | | | | 8 | 0, 30, 60, 240 | min | 240 | min |
| | | | | 9 | 15, 45, 60 | min | 60 | min |
| | | | | 10 | 30, 60, 90 | min | 90 | min |
| | | | | 11 | 45, 90, 120 | min | 120 | min |
| | | | | 12 | 0, 15, 30, 90 | min | 90 | min |

### Results

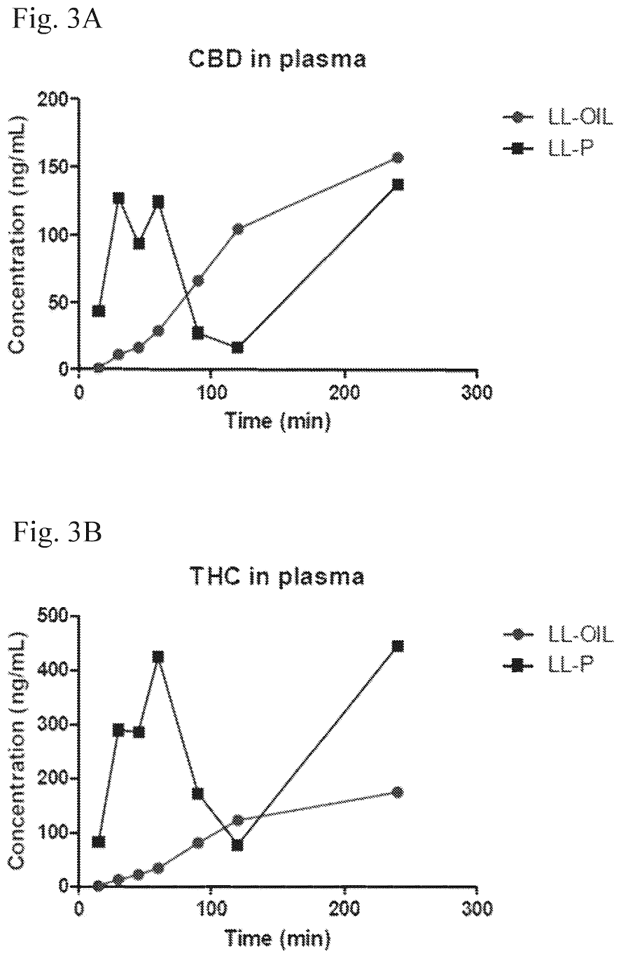

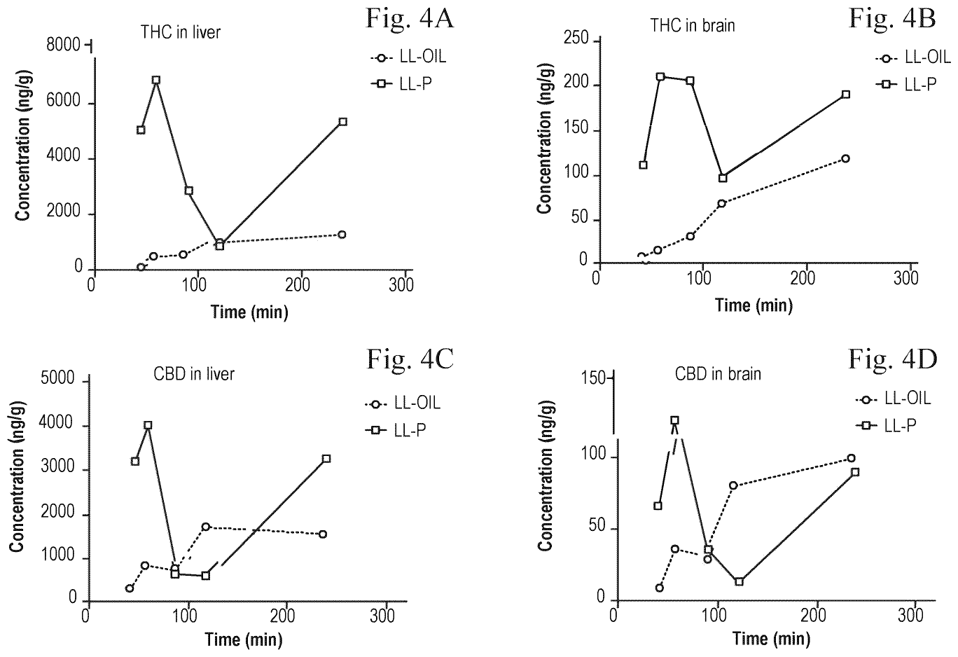

PK analyses of CBD and THC in plasma and selected organs with the two types of compositions are shown in Table 11, and FIGS. 3A-3B (plasma) and FIGS. 4A-4D (liver and brain).

TABLE 11
PK analysis of CBD and THC in plasma, brain and liver
General PK parameters:

| | | CBD | CBD | THC | THC |
|---|---|---|---|---|---|
| | | LL-P | LL-OIL | LL-P | LL-OIL |
| | | PLASMA | PLASMA | PLASMA | PLASMA |
| Dose Amount | mg | 6.5 | 6.5 | 5.6 | 5.6 |
| Dosage | mg/kg | 15.7 | 15.7 | 13.5 | 13.5 |
| Cₘₐₓ (obs) | ng/ml | 137.0 | 156.6 | 444.4 | 174.6 |
| Tₘₐₓ (obs) | hr | 4.0 | 4.0 | 4.0 | 4.0 |
| AUC (0-4) | ng-hr/ml | | | | |
| (obs area) | | | | | |
| | | THC | THC | CBD | CBD |
| | | LL-P | LL-OIL | LL-P | LL-OIL |
| | | BRAIN | BRAIN | BRAIN | BRAIN |
| Dose Amount | mg | 5.6 | 5.6 | 6.5 | 6.5 |
| Dosage | mg/kg | 13.5 | 13.5 | 15.7 | 15.7 |
| Cₘₐₓ (obs) | ng/g | 206.9 | 115.0 | 122.6 | 95.6 |
| Tₘₐₓ (obs) | hr | 1.0 | 4.0 | 1.0 | 4.0 |
| AUC(0-4) | ng-hr/g | 536.5 | 215.0 | 201.0 | 221.5 |
| (obs area) | | | | | |
| | | THC | THC | CBD | CBD |
| | | LL-P | LL-OIL | LL-P | LL-OIL |
| | | LIVER | LIVER | LIVER | LIVER |
| Dose Amount | ng | 5.6 | 5.6 | 6.5 | 6.5 |
| Dosage | ng/kg | 13.5 | 13.5 | 15.7 | 15.7 |
| Cₘₐₓ (obs) | ng/g | 6828.8 | 1289.0 | 4037.2 | 1604.9 |
| Tₘₐₓ (obs) | hr | 1.0 | 4.0 | 1.0 | 2.0 |
| AUC(0-4) | ng-hr/g | 12982.1 | 3004.1 | 7306.0 | 4184.4 |
| (obs area) | | | | | |

### Conclusions

In plasma, LL-Ps showed a biphasic release profile of THC and CBD with a 1st peak in the release of actives during the first hour and a 2nd peak persisting until termination of the study period. In contrast, LL-OILs showed a lower monophasic release profile for the entire the study period.

The profiles in the liver and brain mimicked the plasma profiles. LL-Ps showed significantly more rapid permeation of both actives compared to LL-OILs, In the brain, LL-Ps CBD Cmax was higher in compared to LL-OIL (122.6 vs. 95.6 ng/g, respectively), the same was true for THC Cmax (206.9 vs. 115 ng/g, respectively). Similar results were observed in the liver.

These results suggest that LL-Ps had superior bioavailability in plasma and tissues over LL-OILs, and further exhibited an advantageous bi-phasic release profile providing an immediate as well as a prolonged actives release.

4.1 Release Profiles of Compositions with Vitamin D3

Advantageous delivery and actives release profiles of the present compositions were further supported in an analogous study comparing PK plasma profiles of the powder compositions with vitamin D3 vs. the oil forms. Nanoemulsions were prepared as per standard protocol using both, lyophilization and spray drying. Table 12 shows that the powder compositions preserved the characteristic features of particle size, etc.

TABLE 12
QC test of the powder composition with Vit.D3¶

| Vit. D powder¤ | QC parameters¤ | |
|---|---|---|
| Powder properties¤ | Fine and white¤ | |
| Vitamin D3 content % (w/w)¤ | 300,000 | IU/g¤ |
| Particle size-nm (in emulsion)¤ | 150-200 | nm¤ |

| | |
|---|---|
| Excipients¤ | Disaccharide, polysaccharide,¤ |
| | natural emulsifier¤ |
| pH level in emulsion¤ | 4.4¤ |
| Time to dissolution (sec)¤ | <90¤ |
| Water content (%)¤ | <2¤ |
| Flowability¤ | Bulk density 0.5 gr/ml¤ |
| | Tap density 0.7 gr/ml¤ |
| | Angle of repose 45°¤ |

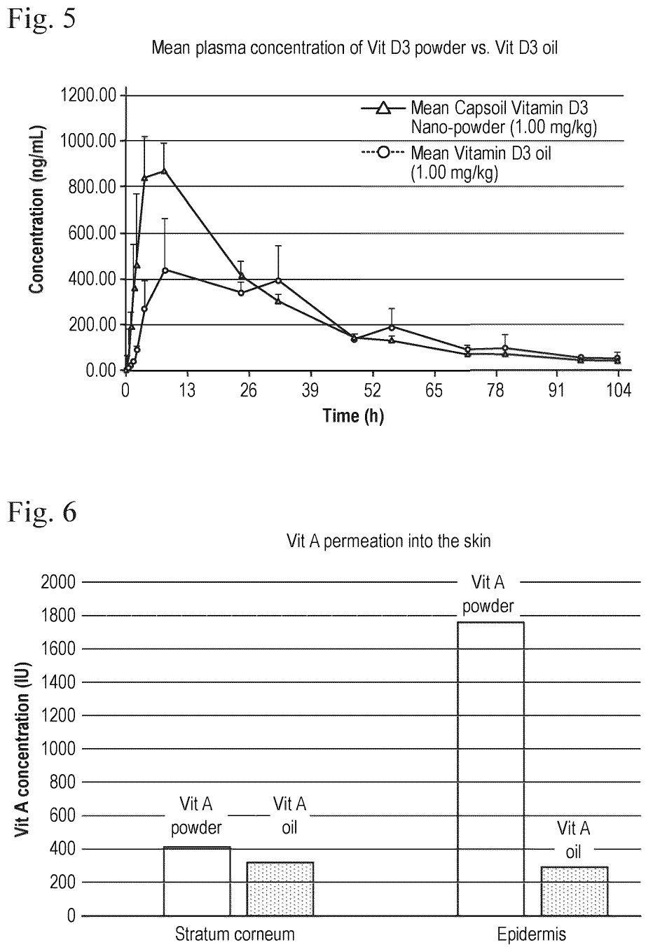

PK analyses were performed in rat plasma (N=9) upon administration of a single oral dose of cholecalciferol (1 mg/kg body weight). Blood samples were collected at 0, 0.25, 0.5, 1, 1.5, 2, 4, 8, 24, 32, 48, 56, 72, 80, 96 and 104 h (4 days). Steady-state cholecalciferol concentrations in plasma were measured by gas-liquid chromatography. Parameters were compared after subtraction of Baseline concentrations and using Baseline concentrations as a covariate. The results are shown in FIG. 6.

The results show that vitamin D3 release from the powder compositions peaked rapidly reaching at a double concertation in plasma relatively to the oil form, and further remained at a lower steady state concertation for at least 60 h (3 days). The bioavailability of vitamin D3 in the powder form as reflected in AUC (area under curve) was higher by 20%, and the half-life was longer by 15% (p<0.05) than in the oil form.

Overall, the results suggest improved delivery and bioavailability of lipophilic actives in the powder compositions of the invention.

### Example 5: Compositions Incorporated into PVA Films

The experiment explored compatibility of the compositions of the invention with topical dosage forms such as PVA dermal patches. Powders containing 30-50% oil were reconstituted in TDW to 0.5% (w/w). PVA solution (8%) and the nanoemulsion were mixed in proportions of 4% and 0.5%, respectively. Samples were casted into aluminum mold and dried at 38° C. for 24 h. Samples' specifications are listed in Table 13.

The produced film (2*1 cm2, ~100 μm thick) was dissolved in TDW at 37° C. and analyzed for oil content. Estimates of particles size before and after release from the film are show in Table 14.

TABLE 16
Samples specifications

| | Nanoemulsion | | | |
|---|---|---|---|---|
| | addition | Actual PVA | Sample size | Dry weight |
| #sample | (g) | conc. | (g) | (g) |
| 1 | | 8.0% | 2.5 | 0.20 |
| 2 | 2.1 | 7.6% | 4.2 | 0.34 |
| 3 | 2.1 | 7.3% | 4.2 | 0.32 |
| 4 | 2.1 | 6.9% | 4.2 | 0.31 |
| 5 | 2.1 | 6.6% | 4.2 | 0.28 |
| 6 | 2.1 | 6.3% | 4.2 | 0.30 |

TABLE 14
Estimates of particle size

| | Average particle size in | Average particle size |
|---|---|---|
| #sample | the pre-formulation | after release from PVA |
| 1 | 187 nm | 207 nm |
| 2 | 187 nm | 210 nm |
| 3 | 187 nm | 208 nm |
| 4 | 187 nm | 214 nm |
| 5 | 187 nm | 202 nm |
| 6 | 187 nm | 202 nm |

The results show that the particle size was maintained in the PVA formulations. Upon drying, the solid particles were evenly fixed in the polymerized film to create a solid-in-solid dispersion. Upon dissolution, the particles were completely released from the polymer, while maintaining the particle size.

Nanometric particle size has a significant impact on the surface area and the permeation rate of actives through biological membranes. In view of that, the finding that the particle size was maintained through various manipulations is particularly important; this is despite the exposure to polar environment (PVA film), temperature and drying. Stability of this structure can be attributed to the unique structure of the nanoparticulate composition of the invention.

Overall, the results suggest that the present powder compositions can be incorporated into polymeric dermal films, thus providing an innovative approach to dermal delivery of actives. Dermal films are particularly advantageous dosage forms for prolonged delivery of substances through the skin. Natural humidity of the skin causes the film to dissolve and slowly release the nanoparticulate lipophilic active embedded in the film until complete dissolution of the film and permeation of the active into the epidermal layers.

### Example 6: Enhanced Permeation into the Skin

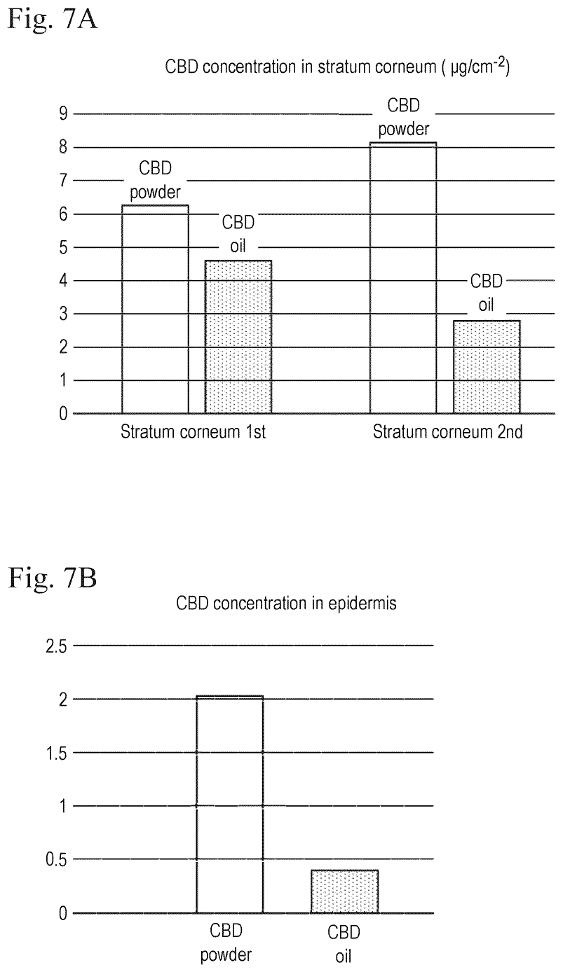

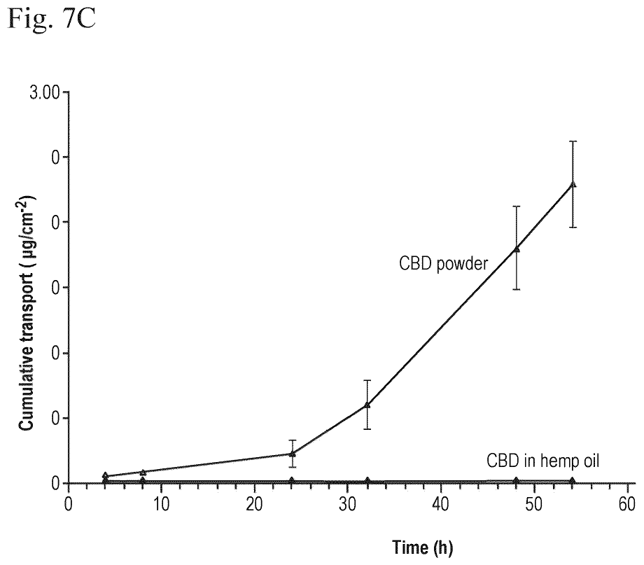

Permeation into the skin was studied comparing the compositions with Vitamin A and CBD in the respective powder and oil forms in ex vivo model of human skin. The results are shown in FIG. 7 and FIGS. 8A-8C, for Vitamin A and CBD, respectively.

The results show that the powder compositions have a significantly enhanced permeation into various layers of the skin compared to the respective oil forms.

For vitamin A for example, the permeation into the epidermis was 6-fold higher with the powder composition than with the respective oil form (FIG. 7). For CBD, the permeation of active in the powder form was slightly higher into the 1st outermost layer of the stratum corneum compared to the oil form and about 4-fold higher into the 2nd layer of the stratum corneum (FIG. 7A), yielding about 10-fold higher permeation of active in the powder form into the epidermis (FIG. 7B), and a significantly higher cumulative transport of active in the powder form into the skin, overall (FIG. 7C).

More generally, the results suggest that the powder compositions of the invention are superior in terms of topical and dermal delivery and bioavailability of actives over the analogous oil forms.

### ANNEX A

Main categories of cosmetic products according to the FDA
01. Baby Products
02. Bath Preparations
03. Eye Makeup Preparations
04. Fragrance Preparations
05. Hair Preparations (non-coloring)
06. Hair Coloring Preparations
07. Makeup Preparations (not eye)
08. Manicuring Preparations
09. Oral Hygiene Products
10. Personal Cleanliness
11. Shaving Preparations
12. Skin Care Preparations (Creams, Lotions, Powders, and Sprays)
13. Suntan Preparations.
   List of Cosmetic Products within these Categories

### 01. Baby Products

a. Baby Shampoos
b. Lotions, Oils, Powders, and Creams
c. Other Baby Products

### 02. Bath Preparations

a. Bath Oils, Tablets, and Salts
b. Bubble Bath
c. Bath Capsules
d. Other Bath Preparations

### 03. Eye Makeup Preparations

a. Eyebrow Pencil
b. Eyeliner
c. Eye Shadow
d. Eye Lotion
e. Eye Makeup Remover
f. Mascara
g. Other Eye Makeup Preparations

### 04. Fragrance Preparations

a. Cologne and Toilet Waters
b. Perfumes
c. Powders (dusting and talcum, excluding aftershave talc)
d. Sachets
e. Other Fragrance Preparations
   05. Hair Preparations (non-coloring)
a. Hair
b. Hair Spray (aerosol fixatives)
c. Hair Straighteners
d. Permanent Waves
e. Rinses (non-coloring)
f. Shampoos (non-coloring)
g. Tonics, Dressings, and Other Hair Grooming Aids
h. Wave Sets
i. Other Hair Preparations

### 06. Hair Coloring Preparations

a. Hair Dyes and Colors (all types requiring caution statements and patch tests)
b. Hair Tints
c. Hair Rinses (coloring)
d. Hair Shampoos (coloring)
e. Hair Color Sprays (aerosol)
f. Hair Lighteners with Color
g. Hair Bleaches
h. Other Hair Coloring Preparations
   07. Makeup Preparations (not eye)
a. Blushers (all types)
b. Face Powders
c. Foundations
d. Leg and Body Paints
e. Lipstick
f. Makeup Bases
g. Rouges
h. Makeup Fixatives
i. Other Makeup Preparations

### 08. Manicuring Preparations

a. Basecoats and Undercoats
b. Cuticle Softeners
c. Nail Creams and Lotions
d. Nail Extenders
e. Nail Polish and Enamel
f. Nail Polish and Enamel Removers
g. Other Manicuring Preparations

### 09. Oral Hygiene Products

a. Dentifrices (aerosol, liquid, pastes, and powders)
b. Mouthwashes and Breath Fresheners (liquids and sprays)
c. Other Oral Hygiene Products

### 10. Personal Cleanliness

a. Bath Soaps and Detergents
b. Deodorants (underarm)
c. Douches
d. Feminine Deodorants
e. Other Personal Cleanliness Products

### 11. Shaving Preparations

a. Aftershave Lotion
b. Beard Softeners
c. Men's Talcum
d. Preshave Lotions (all types)
e. Shaving Cream (aerosol, brushless, and lather)
f. Shaving Soap (cakes, sticks, etc.)
g. Other Shaving Preparations

### 12. Skin Care Preparations (Creams, Lotions, Powders, and Sprays)

a. Cleansing (cold creams, cleansing lotions, liquids, and pads)
b. Depilatories
c. Face and Neck (excluding shaving preparations)
d. Body and Hand (excluding shaving preparations)
e. Foot Powders and Sprays
f. Moisturizing
g. Night
h. Paste Masks (mud packs)
i. Skin Fresheners
j. Other Skin Care Preparations

### 13. Suntan Preparations

a. Suntan Gels, Creams, and Liquids
b. Indoor Tanning Preparations
c. Other Suntan Preparations

### Major Natural Oils

Coconut oil, an oil high in saturated fat
Corn oil, an oil with little odor or taste
Cottonseed oil, an oil low in trans-fats
Canola oil, (a variety of rapeseed oil)
Olive oil
Palm oil, the most widely produced tropical oil
Peanut oil (ground nut oil)
Safflower oil
Sesame oil, including cold pressed light oil and hot pressed darker oil
Soybean oil, produced as a byproduct of processing soy meal
Sunflower oil

### Natural Nut Oils

Almond oil
Cashew oil,
Hazelnut oil
Macadamia oil, has no trans-fats, and a good balance omega-3/omega-6
Pecan oil
Pistachio oil
Walnut oil
Nutrient rich oils
Amaranth oil, high in squalene and unsaturated fatty acids
Apricot oil
Argan oil, a food oil from Morocco
Artichoke oil, extracted from the seeds of Cynara cardunculus
Avocado oil
Babassu oil, a substitute for coconut oil
Ben oil, extracted from the seeds of Moringa oleifera
Borneo tallow nut oil, extracted from the fruit of Shorea
Buffalo gourd oil, extracted from the seeds of Cucurbita foetidissima
Carob pod oil (Algaroba oil)
Coriander seed oil
False flax oil made of the seeds of Camelina sativa
Grape seed oil
Hemp oil, a high quality food oil
Kapok seed oil
Lallemantia oil, extracted from the seeds of Lallemantia iberica
Meadowfoam seed oil, highly stable with over 98% long-chain fatty acids
Mustard oil (pressed)
Okra seed oil, extracted from the seed of Hibiscus esculentus
Perilla seed oil, high in omega-3 fatty acids
Pequi oil, extracted from the seeds of Caryocar brasiliensis
Pine nut oil, an expensive food oil from pine nuts
Poppyseed oil
Prune kernel oil, a gourmet cooking oil.
Pumpkin seed oil, a specialty cooking oil
Quinoa oil, similar to corn oil
Ramtil oil, pressed from the seeds of Guizotia abyssinica (Niger pea)
Rice bran oil
Tea oil (Camellia oil)
Thistle oil, pressed from the seeds of Silybum marianum.

### Natural Sugars

Beet sugar, white and granulated sugar
Cane sugar, white refined or brown sugar
Brown sugar, granulated cane sugar that has molasses (dark and light brown)
Demerara sugar, a type of raw cane sugar
Fructose, fruit sugar twice as sweet as refined cane sugar
Fruit sweetener (liquid and solid) made from grape juice concentrate blended with rice syrup
Jaggery (palm sugar, gur), made from the reduced sap of either the sugar palm or the palmyra palm
Maple sugar, much sweeter than white sugar and has fewer calories
Muscovado (Barbados) sugar, a raw cane sugar similar to brown sugar
Piloncillo (panela, panocha), another type of a raw cane sugar
Rock sugar (Chinese rock sugar), a lightly caramelized cane sugar
Sucanat: juice from organically grown sugarcane turned into granular sugar
Turbinado sugar, raw cane sugar crystals derived from sugarcane
White refined sugar (granulated sugar, table sugar, sucrose) derived from sugarcane or sugar beets

### Natural Polysaccharides

Starch, generally a polymer consisting of two amylose (normally 20-30%) and amylopectin (normally 70-80%) primarily found in cereal grains and tubers like corn (maize), wheat, potato, tapioca, and rice.
Kaempferia rotunda and Curcuma xanthorrhiza essential oils that are enriched in cassava starch-based polysaccharide
Maltodextrin, a polysaccharide produced from vegetable starch
Alginate, a naturally occurring anionic polymer obtained from brown seaweed, also used in various pharmaceutical preparations such as gaviscon, bisodol, and asilone
Carrageenans, water-soluble polymers with a linear chain of partially sulfated galactans
Pectins, a group of plant-derived polysaccharides.
Agars, hydrophilic colloids that have the ability to form reversible gels
Chitosan, a promising group of natural polymers with characteristics such as biodegradability, chemical inertness, biocompatibility, high mechanical strength
Gums, polymer preparations used for their texturizing capabilities
Certain cellulose derivative forms, predominantly four are used in the food industry: hydroxypropyl cellulose (HPC), hydroxypropyl methylcellulose (HPMC), carboxymethylcellulose (CMC), or methylcellulose (MC).

### FDA Approved Emulsifiers

lecithin and lecithin derivatives
glycerol fatty acid esters
hydroxycarboxylic acid and fatty acid esters
lactylate fatty acid esters
polyglycerol fatty acid esters
ethylene or propylene glycol fatty acid esters
ethoxylated derivatives of monoglycerides

### Natural and Nature-Identical Colorants Allowed in the EU and the USA

Curcumin (Turmeric
Riboflavin
Cochineal, Cochineal extract, carminic acid, carmines
Chlorophyll (in) s copper complexes chlorophyll (in) s
Caramel
Vegetable carbon
Carrot oil, β-carotene
Annatto, bixin, norbixin
Paprika extract
Lycopene
β-Apo-8′-carotenal
Ethyl ester of β-apo-8′-carotenoic acid
Lutein
Canthaxanthin
Beetroot red
Anthocyanins
Cottonseed flour
Vegetable juice
Saffron

### Acidulants and Other Preservatives

Lactic acid, acetic acid and other acidulants, alone or in conjunction with other preservatives such as sorbate and benzoate
Malic and tartaric (tartric) acids
Citric acid
Ascorbic acid/vitamin C, isoascorbic isomer, erythorbic acid and their salts
Lipophilic preservatives
Benzoic acid in the form of its sodium salt
Sorbic acid and potassium sorbate, specifically for mold and yeast inhibition
Lipophilic arginine esters, a more recent group of compounds.

1. A water-dispersible powder for cosmetic applications constituting a composite matter comprising micrometric particles of 100 μm to 300 μm comprising at least one sugar, at least one polysaccharide, at least one surfactant and an oil component comprised of at least one liquid cosmetic oil and/or at least one cosmetic lipophilic active ingredient comprised or dissolved in said oil,
said micrometric particles having the oil component entrapped in the form of a plurality of lipophilic nanospheres of average size of 50 nm to 900 nm with an encapsulation efficiency of at least 97%,
wherein the at least one cosmetic lipophilic active ingredient is distributed inside and outside the lipophilic nanospheres,
thereby providing the powder an improved stability and an improved delivery of actives and oils though at least one layer of the skin.

2. The powder of claim 1, wherein the improved delivery accounts for a prolonged penetration and/or permeation of the at least one cosmetic lipophilic active ingredient and/or the at least one cosmetic oil through at least one layer of the skin.

3. The powder of claim 1, wherein the at least one cosmetic lipophilic active ingredient is selected from a peptide, a polysaccharide, a phenolic substance, an aromatic substance, an enzyme, a nucleic acid, a phytochemical, a vitamin, an antioxidant, an essential oil, an extract of an animal or a plant, or any combination thereof.

4. The powder of claim 1, wherein the at least one cosmetic lipophilic active ingredient is selected from a UV protection agent, a moisturizing agent, a whitening agent, a tanning agent, an anti-wrinkling agent, an elasticity promoting agent, an antiaging agent, a skin exfoliation agent, or a combination thereof.

5. The powder of claim 1, wherein the at least one cosmetic oil is selected from a phytochemical, a vitamin, an antioxidant, a phenolic oil, and aromatic oil, an essential oil, an extract of an animal or a plant, or any combination thereof.

6. The powder of claim 1, wherein the improved stability accounts for stability of the at least one cosmetic lipophilic active ingredient for at least 2 years at room temperature.

7. The powder of claim 1, wherein the at least one cosmetic oil is selected from the groups of acylglycerols, mono-(MAG), di-(DAG) and triacylglycerols (TAG), medium-chain triglycerides (MCT), long chain triglycerides (LCT), saturated or unsaturated fatty acids.

8. The powder of claim 1, wherein:
the at least one sugar is selected from trehalose, sucrose, mannitol, lactitol and lactose, and/or
the at least one polysaccharide is selected from maltodextrin and carboxymethyl cellulose (CMC), and/or
wherein the at least one surfactant is selected from ammonium glycyrrhizinate, pluronic F-127 and pluronic F-68.

9. The powder of claim 1, wherein the at least one surfactant is selected from a monoglyceride, a diglycerine, a glycolipid, a lecithin, a fatty alcohol, a fatty acid, a sucrose fatty acid ester (sugar ester) or a mixture thereof.

10. A topical composition comprising an effective amount of the powder of claim 1 and a cosmetic carrier and/or a cosmetic excipient.

11. A cosmetic material comprising the powder of claim 1, the cosmetic material being in the form of a powder, a stick, a lotion, a gel, a cream, an emulsion, or a dermal patch.

12. A non-therapeutic method for cosmetic treatment of a subject, the method comprising topical administering to the subject an effective amount of the powder of claim 1, optionally in a cosmetic carrier and/or excipient.

13. The powder of claim 1, further comprising at least one additional cosmetic oil which is solid or semisolid at room temperature.

14. The powder of claim 1, wherein the at least one sugar is selected from the groups of oligo-, di-, monosaccharides and polyols.

15. The powder of claim 1, wherein the at least one polysaccharide is a natural polysaccharide selected from the groups of fructans, galactans, pectins, starch, alginates, carreageenans and xantham gum.

16. The powder of claim 1, wherein the at least one surfactant is a nonionic or ionic surfactant or a natural emulsifier.

17. The powder of claim 1, wherein the at least one surfactant is a nonionic surfactant selected from the groups of polyol esters, polyoxyethylene esters and poloxamers.

18. The powder of claim 1, wherein the improved stability accounts for preservation of the lipophilic nanospheres' size upon dispersion of the powder in water and in high osmolarity solutions.
